SEQUENCE LISTING (74 sequences)

SEQ ID NO: 1 - protein, 26 aa, Artificial Sequence
  Synthetic
MAVPPTYADLGKSARDVFTKGYGFGL

SEQ ID NO: 2 - protein, 25 aa, Artificial Sequence
  Synthetic
  X (21)..(21): wherein X is any amino acid other than Glycine
  X (23)..(23): wherein X is any amino acid other than Glycine
  X (25)..(25): wherein X is any amino acid other than Glycine
MAVPPTYADLGASAADVFTKXYXFX

SEQ ID NO: 3 - protein, 5 aa, Artificial Sequence
  Synthetic
  X (2)..(4): wherein X is any amino acid
GXXXG

SEQ ID NO: 4 - protein, 24 aa, Artificial Sequence
  Synthetic
  X (21)..(21): wherein X is any amino acid other than Glycine
  X (23)..(23): wherein X is any amino acid other than Glycine
MAVPPTYADLGASAADVFTKXYXF

SEQ ID NO: 5 - protein, 23 aa, Artificial Sequence
  Synthetic
  X (21)..(21): wherein X is any amino acid other than Glycine
  X (23)..(23): wherein X is any amino acid other than Glycine
MAVPPTYADLGASAADVFTKXYX

SEQ ID NO: 6 - protein, 22 aa, Artificial Sequence
  Synthetic
  X (21)..(21): wherein X is any amino acid other than Glycine
MAVPPTYADLGASAADVFTKXY

SEQ ID NO: 7 - protein, 21 aa, Artificial Sequence
  Synthetic
  X (21)..(21): wherein X is any amino acid other than Glycine
MAVPPTYADLGASAADVFTKX

SEQ ID NO: 8 - protein, 20 aa, Artificial Sequence
  Synthetic
MAVPPTYADLGASAADVFTK

SEQ ID NO: 9 - protein, 20 aa, Artificial Sequence
  Synthetic
MAVPPTYADLGASAADVFTK

SEQ ID NO: 10 - protein, 24 aa, Artificial Sequence
  Synthetic
MAVPPTYADLGKSARDVFTKGYGF

SEQ ID NO: 11 - protein, 23 aa, Artificial Sequence
  Synthetic
MAVPPTYADLGKSARDVFTKGYG

SEQ ID NO: 12 - protein, 22 aa, Artificial Sequence
  Synthetic
MAVPPTYADLGKSARDVFTKGY

SEQ ID NO: 13 - protein, 21 aa, Artificial Sequence
  Synthetic
MAVPPTYADLGKSARDVFTKG

SEQ ID NO: 14 - protein, 17 aa, Artificial Sequence
  Synethetic
MRQIKIWFQNRRMKWKK

SEQ ID NO: 15 - protein, 7 aa, Artificial Sequence
  Sythnetic
HAIYPRH

SEQ ID NO: 16 - protein, 7 aa, Artificial Sequence
  Synthetic
HRPYIAH

SEQ ID NO: 17 - DNA, 1993 bp, Homo sapiens
attagcgcagggacctccgggccacagctcagagaatcggaaggcctcctcccccttcccgagcgctgccactggggccgaggtttccagcaagaacccgcgtgtccctgcgcacgcacacacggtgcacacgtcagtccggcgcctccccgtgccccgactcacgcaggtcctcccgcgcgcccgcaacacgcccgcaggctcctgtgtctgctgccggggcagcggggcccggaaggcagaagatggctgtgccacccacgtatgccgatcttggcaaatctgccagggatgtcttcaccaagggctatggatttggcttaataaagcttgatttgaaaacaaaatctgagaatggattggaatttacaagctcaggctcagccaacactgagaccaccaaagtgacgggcagtctggaaaccaagtacagatggactgagtacggcctgacgtttacagagaaatggaataccgacaatacactaggcaccgagattactgtggaagatcagcttgcacgtggactgaagctgaccttcgattcatccttctcacctaacactgggaaaaaaaatgctaaaatcaagacagggtacaagcgggagcacattaacctgggctgcgacatggatttcgacattgctgggccttccatccggggtgctctggtgctaggttacgagggctggctggccggctaccagatgaattttgagactgcaaaatcccgagtgacccagagcaactttgcagttggctacaagactgatgaattccagcttcacactaatgtgaatgacgggacagagtttggcggctccatttaccagaaagtgaacaagaagttggagaccgctgtcaatcttgcctggacagcaggaaacagtaacacgcgcttcggaatagcagccaagtatcagattgaccctgacgcctgcttctcggctaaagtgaacaactccagcctgataggtttaggatacactcagactctaaagccaggtattaaactgacactgtcagctcttctggatggcaagaacgtcaatgctggtggccacaagcttggtctaggactggaatttcaagcataaatgaatactgtacaattgtttaattttaaactattttgcagcatagctaccttcagaatttagtgtatcttttaatgttgtatgtctgggatgcaagtattgctaaatatgttagccctccaggttaaagttgattcagctttaagatgttacccttccagaggtacagaagaaacctatttccaaaaaaggtcctttcagtggtagactcggggagaacttggtggcccctttgagatgccaggtttcttttttatctagaaatggctgcaagtggaagcggataatatgtaggcactttgtaaattcatattgagtaaatgaatgaaattgtgatttcctgagaatcgaaccttggttccctaaccctaattgatgagaggctcgctgcttgatggtgtgtacaaactcacctgaatgggacttttttagacagatcttcatgacctgttcccaccccagttcatcatcatctcttttacaccaaaaggtctgcagggtgtggtaactgtttcttttgtgccattttggggtggagaaggtggatgtgatgaagccaataattcaggacttattccttcttgtgttgtgtttttttttggcccttgcaccagagtatgaaatagcttccaggagctccagctataagcttggaagtgtctgtgtgattgtaatcacatggtgacaacactcagaatctaaattggacttctgttgtattctcaccactcaatttgttttttagcagtttaatgggtacattttagagtcttccattttgttggaattagatcctccccttcaaatgctgtaattaacaacacttaaaaaacttgaataaaatattgaaacctcatccttcttctgttgtctttattaataaaatataaataaac

SEQ ID NO: 18 - DNA, 19 bp, Artificial Sequence
  Synthetic
acacuaggcaccgagauua

SEQ ID NO: 19 - DNA, 19 bp, Artificial Sequence
  Synthetic
gggcuauggauuuggcuua

SEQ ID NO: 20 - DNA, 19 bp, Artificial Sequence
  Synthetic
gcuuggucuaggacuggaa

SEQ ID NO: 21 - DNA, 19 bp, Artificial Sequence
  Synthetic
aagcugaccuucgauucau

SEQ ID NO: 22 - DNA, 19 bp, Artificial Sequence
  Synthetic
gaaugacgggacagaguuu

SEQ ID NO: 23 - DNA, 19 bp, Artificial Sequence
  Synthetic
ucggaauagcagccaagua

SEQ ID NO: 24 - DNA, 19 bp, Artificial Sequence
  Synthetic
cucuucuggauggcaagaa

SEQ ID NO: 25 - DNA, 20 bp, Artificial Sequence
  Synthetic
gaauagcagccaaguaucag

SEQ ID NO: 26 - DNA, 19 bp, Artificial Sequence
  Synthetic
uaagccaaauccauagccc

SEQ ID NO: 27 - DNA, 19 bp, Artificial Sequence
  Synthetic
uuccaguccuagaccaagc

SEQ ID NO: 28 - DNA, 19 bp, Artificial Sequence
  Synthetic
ugauacuuggcugcuauuc

SEQ ID NO: 29 - DNA, 19 bp, Artificial Sequence
  Synthetic
  modified_base (5)..(5): um
  modified_base (8)..(8): gm
  modified_base (13)..(13): gm
  modified_base (17)..(17): um
acacuaggcaccgagauua

SEQ ID NO: 30 - DNA, 19 bp, Artificial Sequence
  Synthetic
  modified_base (6)..(6): um
  modified_base (16)..(16): gm
uaaucucggugccuagugu

SEQ ID NO: 31 - DNA, 19 bp, Artificial Sequence
  Synthetic
uaaucucggugccuagugu

SEQ ID NO: 32 - DNA, 22 bp, Artificial Sequence
  Synthetic
  modified_base (4)..(4): um or uracil
  modified_base (14)..(14): gm
  modified_base (17)..(17): um or uracil
gaauagcagccaaguaucagtt

SEQ ID NO: 33 - DNA, 21 bp, Artificial Sequence
  Synthetic
  modified_base (7)..(7): um
  modified_base (13)..(13): gm
ugauacuuggcugcuauuctt

SEQ ID NO: 34 - DNA, 52 bp, Artificial Sequence
  Synthetic
gccgatcttggcgcgtccgccgcggatgtcttcaccgcgggctacggctttg

SEQ ID NO: 35 - DNA, 52 bp, Artificial Sequence
  Synthetic
caaagccgtagcccgcggtgaagacatccgcggcggacgcgccaagatcggc

SEQ ID NO: 36 - DNA, 20 bp, Artificial Sequence
  Synthetic
accagaatgcctggaacaac

SEQ ID NO: 37 - DNA, 21 bp, Artificial Sequence
  Synthetic
atcagcaccttgaagaagtgt

SEQ ID NO: 38 - DNA, 21 bp, Artificial Sequence
  Synthetic
ccaagtgctgccgtcattttc

SEQ ID NO: 39 - DNA, 21 bp, Artificial Sequence
  Synthetic
ggctcgcagggatgatttcaa

SEQ ID NO: 40 - DNA, 21 bp, Artificial Sequence
  Synthetic
gacttcaacagcaactcccac

SEQ ID NO: 41 - DNA, 20 bp, Artificial Sequence
  Synthetic
tccaccaccctgttgctgta

SEQ ID NO: 42 - DNA, 27 bp, Artificial Sequence
  Synthetic
ctgattacaaaagaagacatgacagac

SEQ ID NO: 43 - DNA, 20 bp, Artificial Sequence
  Synthetic
aggcaaaaccaaagactcca

SEQ ID NO: 44 - DNA, 20 bp, Artificial Sequence
  Synthetic
caaggcaggtttctgaggag

SEQ ID NO: 45 - DNA, 20 bp, Artificial Sequence
  Synthetic
gacctggtcaccatcagcat

SEQ ID NO: 46 - DNA, 19 bp, Artificial Sequence
  Synthetic
ttcccagcagcacagaaac

SEQ ID NO: 47 - DNA, 20 bp, Artificial Sequence
  Synthetic
aaattccaggtgaaatggca

SEQ ID NO: 48 - DNA, 25 bp, Artificial Sequence
  Synthetic
ctttcctcatgatcctggtaatgat

SEQ ID NO: 49 - DNA, 24 bp, Artificial Sequence
  Synthetic
aatccaaaatccttcctgtccttc

SEQ ID NO: 50 - DNA, 20 bp, Artificial Sequence
  Synthetic
ccctatggagatgacggaga

SEQ ID NO: 51 - DNA, 20 bp, Artificial Sequence
  Synthetic
cccagtgctggagaaattgt

SEQ ID NO: 52 - DNA, 20 bp, Artificial Sequence
  Synthetic
ctatgacttccccgtcctga

SEQ ID NO: 53 - DNA, 20 bp, Artificial Sequence
  Synthetic
tcagaaattgccgcttcttt

SEQ ID NO: 54 - DNA, 19 bp, Artificial Sequence
  Synthetic
ctagagctagagcctgcag

SEQ ID NO: 55 - DNA, 19 bp, Artificial Sequence
  Synthetic
agttagtcacggacaccag

SEQ ID NO: 56 - DNA, 20 bp, Artificial Sequence
  Synthetic
ggatgcctgggagagaatcg

SEQ ID NO: 57 - DNA, 20 bp, Artificial Sequence
  Synthetic
tcgcctgctcttcgaaactg

SEQ ID NO: 58 - DNA, 20 bp, Artificial Sequence
  Synthetic
acacagccaagacatccttc

SEQ ID NO: 59 - DNA, 21 bp, Artificial Sequence
  Synthetic
caagtattcacccctgtcctg

SEQ ID NO: 60 - DNA, 19 bp, Artificial Sequence
  Synthetic
gagaggaccatgaagagga

SEQ ID NO: 61 - DNA, 20 bp, Artificial Sequence
  Synthetic
taaaccaaccagaccatgag

SEQ ID NO: 62 - DNA, 21 bp, Artificial Sequence
  Synthetic
actaatgtgaatgacgggaca

SEQ ID NO: 63 - DNA, 19 bp, Artificial Sequence
  Synthetic
gcattgacgttcttgccat

SEQ ID NO: 64 - DNA, 23 bp, Artificial Sequence
  Synthetic
aatctaccatcctccgtgaaacc

SEQ ID NO: 65 - DNA, 25 bp, Artificial Sequence
  Synthetic
tcagtttagctacccccaagtttaa

SEQ ID NO: 66 - DNA, 18 bp, Artificial Sequence
  Synthetic
cccttccccatttggtct

SEQ ID NO: 67 - DNA, 18 bp, Artificial Sequence
  Synthetic
tggtttcacggaggatgg

SEQ ID NO: 68 - DNA, 19 bp, Artificial Sequence
  Synthetic
tcctccgtgaaaccaacaa

SEQ ID NO: 69 - DNA, 18 bp, Artificial Sequence
  Synthetic
agcgagaagaggggcatt

SEQ ID NO: 70 - DNA, 26 bp, Artificial Sequence
  Synthetic
ccccacaaaccccattactaaaccca

SEQ ID NO: 71 - DNA, 26 bp, Artificial Sequence
  Synthetic
tttcatcatgcggagatgttggatgg

SEQ ID NO: 72 - protein, 26 aa, Artificial Sequence
  Synthetic
MAVPPTYADLGASAADVFTAGYGFGL

SEQ ID NO: 73 - DNA, 120 bp, Artificial Sequence
  Synthetic
tcctccgtgaaaccaacaacccgcccaccaatgcccctcttctcgctccgggcccattaaacttgggggtagctaaactgaaactttatcagacatctggttcttacttcagggccatca

SEQ ID NO: 74 - DNA, 120 bp, Artificial Sequence
  Synthetic
tgatggccctgaagtaagaaccagatgtctgataaagtttcagtttagctacccccaagtttaatgggcccggagcgagaagaggggcattggtgggcgggttgttggtttcacggagga

(19) United States
(12) Patent Application Publication
(10) Pub. No.: US 2022/0023381 A1
(43) Pub. Date: Jan. 27, 2022

(54) VDAC INHIBITORS FOR TREATING AUTOIMMUNE DISEASES

(71) Applicants: NATIONAL INSTITUTE FOR BIOTECHNOLOGY IN THE NEGEV LTD., Beer-Sheva (IL); THE UNITED STATES OF AMERICA, AS REPRESENTED BY THE SECRETARY, DEPARTMENT OF HEALTH & HUMAN SERVICES, Bethesda, MD (US)

(72) Inventors: Varda SHOSHAN-BARMATZ, Omer (IL); Jay CHANG, Bethesda, MD (US); Jeonghan KIM, Andong-si, Gyeongsangbuk-do (KR)

(21) Appl. No.: 17/297,017

(22) PCT Filed: Nov. 26, 2019
(86) PCT No.: PCT/IL2019/051290

§ 371 Date: May 26, 2021

Related U.S. Application Data
(60) Provisional application No. 62/771,211, filed on Nov. 26, 2018.

(51) Int. Cl. A61K 38/17; A61K 31/496; A61K 31/4468; A61K 31/454; A61K 31/438; A61P 37/06

(52) U.S. Cl. CPC A61K 38/1709; A61K 31/496; A61P 37/06; A61K 31/454; A61K 31/438; A61K 31/4468

(57) ABSTRACT

The present invention relates to a method for treating diseases associated with type-1 interferon signaling. Particularly, the present invention is directed to use of specific inhibitors of Voltage-Dependent Anion Channel (VDAC1), such as piperazine- and/or piperidine-derivatives, among others, e.g., peptides and oligonucleotides, for treating an autoimmune disease.

### CROSS REFERENCE TO RELATED APPLICATIONS

This application claims the benefit of priority of U.S. Provisional Patent Application No. 62/771,211 titled “VDAC INHIBITORS FOR TREATING AUTOIMMUNE DISEASES”, filed Nov. 26, 2018, the contents of which is incorporated herein by reference in its entirety.

### FIELD OF THE INVENTION

The present invention relates to methods for treating diseases associated with type-1 interferon signaling. Particularly, the present invention relates to Voltage-Dependent Anion Channel (VDAC1) inhibitors for use in treatment of autoimmune diseases.

### BACKGROUND

An autoimmune disease occurs when the body's immune system attacks and destroys healthy body tissues. There are as many as 80 types of autoimmune diseases, among which systemic lupus erythematosus (SLE), multiple sclerosis (MS) and rheumatoid arthritis (RA) are the most common autoimmune diseases in the northern hemisphere affecting an increasing number of patients. Autoimmune diseases are, thus, an enormous global problem that significantly threatens human health.

Typically, autoimmune diseases are treated with nonspecific immunosuppressive agents, such as glucocorticoids, cyclophosphamide, methotrexate, azathioprine, and cyclosporine, that impede the immune cells from attacking the organs and tissues. However, immunosuppressive agents are often associated with significant side effects, e.g., toxicity and the undesired suppression of the immune system.

Self-DNA and type I interferon signaling play a central role in the pathogenesis of autoimmune diseases. It has been shown that deficiency of Trex1, the major 3′-to-5′ exonuclease in the cytosol, leads to accumulation of cytosolic DNA and activation of cyclic GMP-AMP synthase (cGAS) and interferon signaling, leading to autoimmune disease. In humans, Trex1 mutations have been linked to lupus and Aicardi-Goutieres Syndrome. Recent studies have shown that extracellular release of oxidized mitochondrial DNA (mtDNA) from neutrophils in the form of neutrophil extracellular traps (NETs) and/or extruded oxidized nucleoids is also an important trigger for subsequent interferon signaling and development of lupus-like disease.

Mitochondrial DNA is circular DNA that encodes 37 genes, including subunits of protein complexes essential for oxidative phosphorylation. Although mtDNA is present in thousands of copies per cell, most mtDNA does not exist in a free form, but is packaged into nucleoids, large structures that are tethered to the matrix side of the inner mitochondrial membrane (IMM). Although the mechanism is not entirely clear, mtDNA from stressed mitochondria can be released into the cytosol where it can interact with and activate a large number of immunostimulatory DNA sensors. One of the best characterized DNA sensors is cGAS, which generates cyclic dinucleotide cGAMP upon binding to DNA. cGAMP then engages the stimulator of interferon genes (STING), which triggers type I interferon signaling. The degree of stress that releases mtDNA and activates cGAS can range from apoptosis induced by BCL-2-like protein 4 (BAX) and BCL-2 homologous antagonist/killer (BAK) to modest mtDNA stress induced by deficiency of transcription factor A, mitochondrial (TFAM), which is critical for mtDNA packaging. Another category of mtDNA sensors is the inflammasomes, which are triggered by cellular exposure to the so-called damage-associated molecular patterns (DAMPs), molecules that signal cellular stress or infection and subsequent release of mtDNA. Activated inflammasomes induce inflammation by stimulating the release of inflammatory cytokines such as IL-1β and IL-18. Unlike cGAS, which is expressed in many cell types, the inflammasome pathways are largely restricted to macrophages.

The mechanism by which mtDNA is released into the cytosol is poorly understood. It may involve some type of a gated mechanism, membrane damage or a combination of both. One possible clue comes from purified mitochondria, which releases mtDNA with Ca²⁺ overload. Since Ca²⁺ overload opens the mitochondrial permeability transition pore (PTP) on the IMM and mtDNA release is inhibited with cyclosporin A (CysA), which can block PTP opening, it was postulated that PTP may be required for mtDNA fragment release from purified mitochondria. Assuming that mtDNA passes the IMM in an PTP-dependent manner in cells, not just in purified mitochondria, the passage of mtDNA through the outer mitochondrial membrane (OMM) is still not known.

The voltage-dependent anion channel (VDAC), which is composed of three isoforms (VDAC1, 2 and 3), is the most abundant protein in OMNI and regulates metabolism, inflammasome activation and cell death. Although VDAC is the main OMNI channel for Ca²⁺ influx, which is required for PTP opening, VDAC is not a core component of the PTP. VDAC controls the metabolic cross-talk between mitochondria and the rest of the cell, allowing entry of metabolites including pyruvate, malate, succinate, nucleotides, and NADH into mitochondria and the exit of newly formed molecules, such as ATP and hemes, from mitochondria. VDAC is also involved in cholesterol transport, fluxes of ions and serves as the reactive oxygen species (ROS) transporter and regulating mitochondrial and cytosolic redox states.

VDAC is composed of an amphipathic 26 amino acid long N-terminal α-helix region and membrane-embedded β-barrel. The N-terminal region, which is highly dynamic, is proposed to move within the pore and also to translocate from within the pore to the channel surface. The diameter of the VDAC pore is about 1.5 nm when the N-terminal region is located within the pore and between 3 and 3.8 nm when the N-terminal region is located outside the pore. The pore of the monomer may be too small to allow mtDNA (2 nm diameter) to cross the OMNI, but VDAC is found in a dynamic equilibrium between monomeric and oligomeric states, and the oligomers may form pores significantly larger than that of the monomer.

Thus, there remains an unmet need for improved methods of treating type 1 interferon-mediated diseases, particularly autoimmune diseases, which provide increased efficacy but do not involve broad immune suppression.

### SUMMARY

The present invention provides methods for slowing the progression of or treating an autoimmune disease comprising reducing the expression or activity of VDAC in a subject in need thereof.

The present invention is based in part on the discovery that mitochondrial DNA (mtDNA) released either into the cytosol and/or the extracellular space plays a major role in type-1 interferon signaling. It is now shown that under conditions where cytosolic mtDNA is increased, such as in endonuclease G (EndoG)-deficient fibroblasts, interferon-stimulated gene (ISG) expression is increased. The inventors of the present invention show for the first time that inhibition of VDAC1 expression or VDAC1 activity by various means, e.g., by a specific piperazine derivative known to inhibit VDAC1 oligomerization and designated herein below as “VBIT-4”, significantly reduced both mtDNA release to the cytosol of EndoG^{-/-} fibroblasts and ISG expression in these cells.

It is now further disclosed that mtDNA interacted with VDAC through its N-terminal domain and such interaction increased the formation of VDAC oligomers, the latter process was shown to be important for intracellular mtDNA release.

Unexpectedly, the inventors of the present invention disclose that administration of VBIT-4 to mice having lupus-like disease blocked the development of skin lesions, reduced the weight of the spleen and lymph node, and significantly diminished ISG induction, renal immune complex deposition, serum anti-dsDNA, proteinuria, and cell-free mtDNA. Additionally, the present invention discloses that VBIT-4 inhibited the formation of neutrophil extracellular traps (NETs) by neutrophils obtained from lupus patients, a process known to trigger autoimmunity.

Thus, the present invention provides highly efficient methods for treating type-1 interferon-mediated diseases, particularly autoimmune diseases such as systemic lupus erythematosus, which avoid broad immune suppression. This method may also be effective in treatment of other interferonopathies including, but not limited to, Aicardi-Goutières syndrome (AGS), Retinal vasculopathy with cerebral leukodystrophy (RVCL) and STING-associated vasculopathy, infantile-onset (SAVI).

According to one aspect, the present invention provides a method for slowing the progression of or treating an autoimmune disease or one or more symptoms associated therewith, the method comprising administering to a subject in need of such treatment a pharmaceutical composition comprising a therapeutically effective amount of a VDAC inhibitor.

In some embodiments, the VDAC inhibitor is a compound of the general Formula (I):

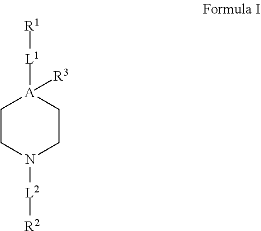

wherein: A is carbon (C) or nitrogen (N); R³ is absent, or is selected from a hydrogen, an unsubstituted or substituted amide or a heteroalkyl group comprising 3-12 atoms apart from hydrogen atoms, wherein at least one of said 3-12 atoms is a heteroatom, selected from nitrogen, sulfur and oxygen; wherein when A is nitrogen (N), R³ is absent; L¹ is absent or is an amino linking group —NR⁴—, wherein R⁴ is hydrogen, a C₁₋₅-alkyl, a C₁₋₅-alkylene or a substituted alkyl —CH₂R, wherein R is a functional group selected from the group consisting of hydrogen, halo, haloalkyl, cyano, nitro, hydroxyl, alkyl, alkenyl, aryl, alkoxyl, aryloxyl, aralkoxyl, alkylcarbamido, arylcarbamido, amino, alkylamino, arylamino, dialkylamino, diarylamino, arylalkylamino, aminocarbonyl, alkylaminocarbonyl, arylaminocarbonyl, alkylcarbonyloxy, arylcarbonyloxy, carboxyl, alkoxycarbonyl, aryloxycarbonyl, sulfo, alkyl sulfonylamido, alkyl sulfonyl, arylsulfonyl, alkylsulfinyl, arylsulfinyl and heteroaryl; R¹ is an aromatic moiety, which is optionally substituted with one or more of Z; Z is independently at each occurrence a functional group selected from the group consisting of, hydrogen, halo, haloalkyl, haloalkoxy, perhaloalkoxy or C₁₋₂-perfluoroalkoxy, cyano, nitro, hydroxyl, alkyl, alkenyl, aryl, alkoxyl, aryloxyl, aralkoxyl, alkylcarbamido, arylcarbamido, amino, alkylamino, arylamino, dialkylamino, diarylamino, arylalkylamino, aminocarbonyl, alkylaminocarbonyl, arylaminocarbonyl, alkylcarbonyloxy, arylcarbonyloxy, carboxyl, alkoxycarbonyl, aryloxycarbonyl, sulfo, alkyl sulfonylamido, alkylsulfonyl, arylsulfonyl, alkylsulfinyl, arylsulfinyl and heteroaryl; L² is a linking group, such that when A is nitrogen (N), L² is a group consisting of 4-10 atoms, apart from hydrogen atoms, optionally forming a ring, whereof at least one of the atoms is nitrogen, said nitrogen forming part of an amide group; and when A is carbon (C), then L² is selected from C₁₋₄ alkylene or a group consisting of 4-10 atoms, apart from hydrogen atoms, optionally forming a ring, whereof at least one of the atoms is nitrogen, said nitrogen forming part of an amide group; and R² is a phenyl or a naphthyl, optionally substituted with a halogen; or an enantiomer, diastereomer, mixture or salt thereof.

According to some embodiments, the compound has the formula selected from the group consisting of formulae 1, 2, 3, 4, 5, 6, 7, 8, 9, 10, and 11.

According to one embodiment, the compound is N-(4-chlorophenyl)-4-hydroxy-3-(4-(4-(trifluoromethoxy)phenyl)-piperazin-1-yl)butanamide (Formula 1), designated throughout the specification VBIT-4.

According to another embodiment, the compound is 1-(4-chlorophenyl)-3-(4-(4-(trifluoromethoxy)phenyl)piperazin-1-yl)pyrrolidine-2,5-dione (Formula 2), designated throughout the specification VBIT-3.

According to a further embodiment, the compound is (1-(naphthalen-2-ylmethyl)-4-(phenylamino)piperidine-4-carbonyl)glycine (Formula 3), designated throughout the specification VBIT-12.

According to some embodiments, the VDAC inhibitor is a peptide derived from or corresponding to amino acids residues 1-26 of human VDAC1 N-terminal domain (SEQ ID NO:1) comprising: (i) one or more mutations compared to SEQ ID NO:1, (ii) a truncation of one or more amino acids compared to SEQ ID NO:1, or a combination thereof.

According to some embodiments, the VDAC inhibitor is a peptide of 1-25 amino acids comprising a contiguous sequence derived from amino acids residues 1-26 of human VDAC1 N-terminal domain comprising the amino acid sequence: MAVPPTYADLGKSARDVFTKXYXFX (SEQ ID NO:2), wherein X is any amino acid other than glycine.

According to certain exemplary embodiments, the peptide comprises an amino acid sequence selected from the group consisting of: SEQ ID Nos.:4-13.

According to some embodiments, the VDAC inhibitor is a VDAC silencing oligonucleotide molecule, or a construct comprising same. Any VDAC silencing oligonucleotide molecule may be used in the methods of the present invention, as long as the oligonucleotide comprises at least 15 contiguous nucleic acids identical to SEQ ID NO:17, to an mRNA molecule encoded by same or to a sequence complementary thereto.

According to certain embodiments, the silencing oligonucleotide comprises a nucleic acid sequence selected from the group consisting of: SEQ ID NO:18; SEQ ID NO:19; SEQ ID NO:20; SEQ ID NO:21; SEQ ID NO:22; SEQ ID NO:23; SEQ ID NO:24; and SEQ ID NO:25.

According to some embodiments, the autoimmune disease is selected from the group consisting of autoimmune diseases involving a systemic autoimmune disorder and autoimmune diseases involving a single organ or single cell-type disorder.

According to additional embodiments, the autoimmune disease involving a systemic autoimmune disorder is selected from the group consisting of systemic lupus erythematosis (SLE), rheumatoid arthritis (RA), Sjogren's syndrome, systemic sclerosis, and bullous pemphigoid. Each possibility represents a separate embodiment of the invention. According to a certain embodiment, the autoimmune disease involving a systemic autoimmune disorder is SLE. According to a further embodiment, the autoimmune disease involving a systemic autoimmune disorder is RA. According to yet further embodiment, the autoimmune disease involving a systemic autoimmune disorder is multiple sclerosis, wherein the subject does not suffer from depression or any other mood disorder.

According to further embodiments, the autoimmune disease involving a single cell-type autoimmune disorder is selected from the group consisting of Hashimoto's thyroiditis, autoimmune hemolytic anemia, autoimmune atrophic gastritis, autoimmune encephalomyelitis, autoimmune orchitis, Goodpasture's disease, autoimmune thrombocytopenia, myasthenia gravis (MG), Graves' disease, primary biliary cirrhosis, membranous glomerulopathy, Aicardi-Goutières syndrome (AGS), Retinal vasculopathy with cerebral leukodystrophy (RVCL) and STING-associated vasculopathy, infantile-onset (SAVI).

According to some embodiments, the pharmaceutical composition is formulated for oral administration route or for parenteral administration route. According to additional embodiments, the pharmaceutical composition is formulated as a solution, suspension, emulsion, tablet, lozenge, powder, spray, foam, cream, gel, or a suppository.

According to some embodiments, the pharmaceutical composition is administered via oral administration route or parenteral administration route. According to additional embodiments, the parenteral administration route is selected from the group consisting of intravenous, subcutaneous, intramuscular, transdermal, topical, intranasal, and intravaginal administration. According to a certain embodiment, the pharmaceutical composition is administered orally.

According to additional embodiments, the pharmaceutical composition further comprises at least one additional active agent known to affect an autoimmune disease.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIGS. 1A-1K are graphs, micrographs, and a heatmap show the effect of endonuclease G (EndoG)-deficiency on increasing cytosolic mtDNA and type I interferon signaling. FIG. 1A shows RNAseq analysis of wild-type and EndoG^{-/-} MEFs presented by heat maps. FIG. 1B shows RNAseq analysis of wild-type and EndoG^{-/-} MEFs presented by RNA read counts visualized by Integrated Genome Viewer (IGV). FIG. 1C shows Real-time PCR analysis of ISG expression in WT and EndoG^{-/-} MEFs. FIG. 1D, shows ISG expression levels measured in EndoG^{-/-} MEFs with stably reintroduced WT EndoG (EndoG^{-/-+WT}). FIG. 1E shows confocal microscopy images of MEFs stained with MitoSOX (mitochondria) and Hoechst (DNA). Mitochondrial ROS levels were visualized in WT and EndoG^{-/-} MEFs (microscopy images, left panels; fluorescence intensity, right panel). Scale bar, 20 μm. FIG. 1F shows ROS levels measured in WT and EndoG^{-/-} MEFs after treatment with Mito-TEMPO (10 μM). FIG. 1G shows ISG expression measured by real-time PCR in WT and EndoG^{-/-} MEFs which were treated with Mito-TEMPO (10 μM). FIG. 1H shows quantification of the cytosolic fraction of mtDNA (cmtDNA) by real-time PCR. Three pairs of primers of the mtDNA D-loop regions were used to quantify cmtDNA from WT and EndoG^{-/-} MEFs. FIG. 1I shows real-time PCR analysis of total mtDNA levels in WT and EndoG^{-/-} MEFs as well as two independently-generated ρ⁰ MEFs (ρ⁰ 1 and ρ⁰ 2), which lack mtDNA, from both WT and EndoG^{-/-} MEFs. FIG. 1J shows ISG expression of the ρ⁰ MEFs determined by western blotting. FIG. 1K shows ISG expression of the ρ⁰ MEFs determined by real-time PCR. All values are presented as the mean±SEM. A two-tailed unpaired Student's t-test was used to evaluate the statistical significance in FIGS. 1C-1D, and 1H-1I; one-way ANOVA with Tukey's post-hoc test for multiple comparisons was used for statistical analysis in FIGS. 1F-1G and 1K. *p<0.05; **p<0.01; ***p<0.001.

FIGS. 2A-2N are graphs, micrographs, and non-limiting illustrations, showing that VDAC is required for release of free intra-mtDNA fragments. FIG. 2A shows ISG expression assessed by real-time PCR in WT and VDAC1/3^{-/-} MEFs. FIG. 2B shows cmtDNA levels as determined after treatment with H₂O₂ (100 μM) in WT and VDAC1/3^{-/-} MEFs by real-time PCR. FIG. 2C shows ISG expression assessed by real-time PCR in WT and VDAC1/3^{-/-} MEFs after the knock-down (KD) of EndoG. FIG. 2D shows ISG expression assessed by real-time PCR in WT and VDAC1/3^{-/-} MEFs after the KD of TFAM. FIGS. 2E-2F show ISG expression as determined after treatment with DIDS (100 μM) in EndoG^{-/-} and TFAM^{KD} MEFs by real-time PCR (FIG. 2E) and western blot (FIG. 2F). FIG. 2G shows mtDNA released from isolated mitochondria from MICU1^{-/-} MEFs as measured by real-time PCR after treatment with DIDS. D-loop, mt-16s and mt-ND4 indicate the three primer pairs used for real-time PCR. FIGS. 2H-2I show cmtDNA (FIG. 2H) and ISG expression (FIG. 2I) levels determined after treatment with VBIT-4 (10 μM) in EndoG^{-/-} MEFs by real-time PCR. FIG. 2J shows VDAC1-dependent release of mtDNA from mtDNA-loaded liposomes. Released mtDNA from liposome was measured by real-time PCR. The released mtDNA is relative to VDAC1-free-liposomes. FIG. 2K shows the distribution of fimtDNA and cmtDNA fragments visualized by Integrated Genome Browser (IGB). Green boxes indicate encoded-mitochondrial gene, and red box indicates the D-loop region. Left panel indicates a schematic diagram of fimtDNA. FIG. 2L shows fragment-size distribution of the fimtDNA plotted to the unique mouse mitochondrial genome sequence only. FIGS. 2M-2N show real-time PCR analysis of the fimtDNA by treatment with 50 nM mito-TEMPO (FIG. 2M) and 100 nM everolimus (FIG. 2N). The fimtDNA in the CSK-supernatant was normalized by mtDNA in the CSK-pellet. Two-tailed unpaired Student's t-test was used to evaluate the statistical significance in FIGS. 2A, 2E, 2H, 2I, 2M and 2N; one-way ANOVA with Tukey's post-hoc test for multiple comparisons was used for statistical analysis in FIGS. 2B-2D, 2G and 2J. *p<0.05; **p<0.01; ***p<0.001; ns, not significant.

FIGS. 3A-3K are non-limiting schematic diagrams, graphs and micrographs showing that mtDNA interacts with VDAC and stabilizes the oligomers. FIG. 3A shows a schematic diagram of channel conductance properties assay by reconstitution of VDAC into a planar lipid bilayer (PLB). FIGS. 3B-3C show the inhibition of VDAC1 channel conductance by mtDNA after prior exposure to high voltage (60 mV). Full length of VDAC1 was purified and reconstituted into an azolectin-planar lipid bilayer membrane. Representative current traces obtained at the indicated voltage with bilayer-reconstituted VDAC1 before and 15 minutes after the addition of mtDNA in the direction of cis (FIG. 3B) or trans (FIG. 3C) at +10 mV and +40 mV. FIG. 3D shows the percentage inhibition of bilayer reconstituted VDAC1 single channel steady state current measured at ±10 mV and ±40 mV upon addition to the cis side the indicated concentrations of mtDNA. (▪) and (◯) indicate recording at positive and negative voltages, respectively. FIG. 3E shows channel conductance by mtDNA on VDAC1ΔN. FIG. 3F is a schematic diagram of VDAC oligomerization showing that in the oligomerized state, the N-terminal region of VDAC1 (red) translocates into the large oligomer pore. The positively charged amino acid residues (+: K12, R15, K20) in the N-terminus region form a positively charged ring around the large pore that can interact with mtDNA and facilitate its passage through the OMNI. FIG. 3G shows that mtDNA induced VDAC1 oligomerization. Purified WT VDAC1 (FIG. 3G) was incubated with 60 nM mtDNA fragment with EGS (100 The oligomerization was determined by western blotting using VDAC1 antibody. FIG. 3h shows quantitative analysis of trimers, tetramers and multimers. FIG. 3I shows the peptide sequence of VDAC1 N-terminal 26 amino acid. The positively charged amino acids were mutated to alanine (A: red color). FIG. 3J shows the interaction of mtDNA fragments with VDAC1 WT and alanine mutant of N-terminal 26 peptide. FIG. 3K shows the ISG expression levels measured in WT and alanine mutant MEFs by real-time PCR. All values are presented as the mean±SEM. A two-tailed unpaired Student's t-test was used to evaluate the statistical significance in FIG. 3J-3K; *p<0.05; **p<0.01; ***p<0.001; ns, not significant.

FIGS. 4A-4H are graphs and micrographs showing the regulation of ISG expression levels by outer mitochondrial membrane-associated proteins, VDAC, Bax/Bak. FIG. 4A shows cmtDNA levels in WT and Bax/Bak−/− MEFs. FIG. 4B shows ISG expression levels were measured in WT, Bax/Bak−/−, and EndoG-knocking down in Bax/Bak−/− MEFs by RT-qPCR. FIGS. 4C-4D shows cmtDNA levels (C) and mtDNA copy number (D) in WT and VDAC1/3−/− MEFs by qPCR. FIG. 4E shows, Ifi44 expression levels of LMTK-1 (WT) and LMEB-4 (ρ0) cells following treatment with 100 μM DIDS. FIGS. 4F-411 show, viral expression in WT and VDAC1/3−/− MEFs infected with HSV-1-RFP (MOI 0.1). Plaque size and red fluorescence intensity were observed under UV microscope (FIG. 4F), percentage of RFP positive cells were determined by FACS (FIG. 4G). The replication kinetics of HSV-1-RFP was determined by virus growth curve. MEFs infected with HSV-1-RFP and harvested at times as shown. Virus titers were then determined in Vero cells (FIG. 4H). All values are presented as the mean±SEM of at least three independent experiments. A two-tailed unpaired Student's t-test was used to evaluate the statistical significance in a-e, g and h. **p<0.01; ***p<0.005; ns, not significant.

FIGS. 5A-5C are sequences alignment, images of 3-dimensional structure, and a graph, showing the function of VDAC N-terminal region. FIG. 5A shows the analysis of VDAC1 N-terminal region sequence in various species. FIG. 5B shows the N-terminal domain structure of VDAC1 WT and mutant as predicted by the SWISS-MODEL server. FIG. 5C shows ISG expression levels were measured in WT and VDAC1ΔN expressing MEFs by real-time PCR. All values are presented as the mean±SEM of three independent experiments. A two-tailed unpaired Student's t-test was used to evaluate the statistical significance in FIG. 5C. *p<0.05; **p<0.01; ***p<0.001; ns, not significant.

FIGS. 6A-6K are graphs and a micrograph showing the role of ROS, Ca²⁺ and VDAC1 oligomerization in mtDNA release. FIGS. 6A-6B show that treatment with Ca²⁺ chelator BAPTA decreased ISG expression in EndoG^{-/-} MEFs or TFAMKD MEFs, but not in VDAC1/3^{-/-} MEFs. FIGS. 6C-6F show that interferon-signaling and mROS level were increased in MICU1^{-/-} MEFs. FIG. 6G shows that treatment with DIDS abrogated ISG induction in these cells. FIGS. 6H-6I show that treatment with CsA of both WT MEFs and mitoplasts decreased mtDNA release, suggesting that in living cells, mtDNA is most likely released from a small subset of unhealthy or damaged mitochondria with opened PTPs. FIGS. 6J-6K show that VBIT-4 did not prevent either Ca²⁺ uptake or PTP opening in purified mitochondria. Taken together, these findings indicate that even though VDAC1 can control PTP opening by serving as the major channel for Ca²⁺ uptake, VDAC1 oligomerization can also promote mtDNA release independent of its functions in Ca²⁺ flux and PTP opening.

FIGS. 7A-7L are images, graphs, and micrographs, showing the protection against lupus-like disease by VDAC oligomerization inhibitor VBIT-4. FIG. 7A shows the inhibition of alopecia in the facial and dorsal areas and erythema in the skin lesions of VBIT-4-treated MRL/lpr mice. The skin of treated mice was stained with hematoxylin and eosin (H&E). FIG. 7B shows the quantification of alopecia of the mice in FIG. 7A. FIG. 7C shows the weight of the spleen and lymph nodes of treated mice at 16 weeks of age. FIG. 7D shows the expression of ISG in the spleen of treated mice. ISG expression levels were measured by real-time PCR. FIG. 7E shows kidney glomeruli of treated mice, stained with antibodies against complement C3 (green) and IgG (red). Nuclei were stained with Hoechst (blue). Scale bar, 50 μm. FIG. 7F shows fluorescence intensity of C3 and IgG in the renal tissue sections of the mice in FIG. 7E. FIGS. 7G-7I show Anti-dsDNA level (FIG. 7G), albumin:creatinine ratio (FIG. 7H), and serum mtDNA level (FIG. 7I) of treated mice. FIG. 7J shows quantification of mitochondrial ROS in the PBMCs of healthy control (HC) or systemic lupus erythematosus (SLE) subjects by fluorometric measurement after 1 h of incubation with MitoSOX. FIG. 7K (Left) Inhibition of spontaneous NET formation of low-density granulocytes (LDG, SLE) by VBIT-4 (5 μM). (Right) Inhibition of A23187-stimulated NET formation of normal-density granulocytes (NDG, SLE) by VBIT-4. Green represents human neutrophil elastase (HNE), and blue represents DNA (Hoechst). Scale bar, 10 μm. FIG. 7L shows NET formation by NDGs either from HC or SLE subjects was measured by SYTOX-PicoGreen plate assay (n=3 in each group). All values are presented as the mean±SEM. Student's t-test was used to evaluate the statistical significance in FIGS. 6B-D, 6F-I, and 6L. Mann-Whitney U test (HC versus SLE) (j) was used. *p<0.05; **p<0.01; ***p<0.001.

FIGS. 8A-8E are graphs and images showing the role of VDAC in a lupus-like disease model. Gene Expression Omnibus (GEO) analysis revealed shows decreased expression EndoG and Tftam gene (FIG. 8A) increased expression of VDAC1/3 (FIG. 8B) and no difference in the expression levels of VDAC2, HSP60, Bak and Bax (FIG. 8C) in healthy control and SLE (Lupus) patients. Raw data were obtained from GEO accession no. GSE13887. FIG. 8D shows the body weight from vehicle and VBIT4 treated mice, at 11 and 16 weeks of age (n=10 in each group). Two-tailed unpaired Student's t-test was used to evaluate statistical significance in FIG. A-D *p<0.05; **p<0.01; ns, not significant. FIG. 8E shows representative photographs of spleen and lymph node from vehicle and VBIT4 treated SLE (Lupus) mice.

FIGS. 9A-9B are non-limiting schematic diagrams showing VDAC oligomerization in mitochondrial membrane as a result of ROS increase and its role in cmtDNA release and in interferon signaling (FIG. 9A) and the inhibitory effect of VBIT-4 on VDAC oligomerization and NETosis in human neutrophils (FIG. 9B).

### DETAILED DESCRIPTION

The present invention is directed to a method for treating diseases mediated by type-1 interferon signaling which comprise administering to a subject in need of such treatment a VDAC inhibitor or a pharmaceutical composition comprising thereof.

The present invention further provides a method for treating autoimmune diseases, slowing the progression of an autoimmune disease or one or more symptoms associated therewith, the method comprising administering to a subject in need of such treatment a VDAC inhibitor or a pharmaceutical composition comprising thereof.

In some embodiments, the method comprises administering a therapeutically effective amount of at least one piperazine- or piperidine-derivative such as disclosed herein below.

### Piperazine Compounds

According to some embodiments, a piperazine- or piperidine-derivative to be used for method of the invention is of general Formula (I):

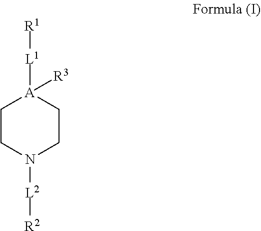

wherein: A is carbon (C) or nitrogen (N); R³ is absent, or is selected from a hydrogen, an unsubstituted or substituted amide or a heteroalkyl group comprising 3-12 atoms apart from hydrogen atoms, wherein at least one of said 3-12 atoms is a heteroatom, selected from nitrogen, sulfur and oxygen; wherein when A is nitrogen (N), R³ is absent; L¹ is absent or is an amino linking group —NR⁴—, wherein R⁴ is hydrogen, a C₁₋₅-alkyl, a C₁₋₅-alkylene or a substituted alkyl —CH₂R, wherein R is a functional group selected from hydrogen, halo, haloalkyl, cyano, nitro, hydroxyl, alkyl, alkenyl, aryl, alkoxyl, aryloxyl, aralkoxyl, alkylcarbamido, arylcarbamido, amino, alkylamino, arylamino, dialkylamino, diarylamino, arylalkylamino, aminocarbonyl, alkylaminocarbonyl, arylaminocarbonyl, alkylcarbonyloxy, arylcarbonyloxy, carboxyl, alkoxycarbonyl, aryloxycarbonyl, sulfo, alkylsulfonylamido, alkyl sulfonyl, aryl sulfonyl, alkylsulfinyl, arylsulfinyl and heteroaryl; preferably R⁴ is hydrogen; R¹ is an aromatic moiety, preferably phenyl, which may be substituted with one or more of Z; Z is independently at each occurrence a functional group selected from hydrogen, halo, haloalkyl, haloalkoxy, perhaloalkoxy or C₁₋₂-perfluoroalkoxy, cyano, nitro, hydroxyl, alkyl, alkenyl, aryl, alkoxyl, aryloxyl, aralkoxyl, alkylcarbamido, arylcarbamido, amino, alkylamino, arylamino, dialkylamino, diarylamino, arylalkylamino, aminocarbonyl, alkylaminocarbonyl, arylaminocarbonyl, alkylcarbonyloxy, arylcarbonyloxy, carboxyl, alkoxycarbonyl, aryloxycarbonyl, sulfo, alkylsulfonylamido, alkylsulfonyl, arylsulfonyl, alkylsulfinyl, arylsulfinyl and heteroaryl; preferably Z is C₁₋₂-perfluoroalkoxy; preferably when A is nitrogen (N) R¹ is a phenyl and Z is trifluoromethoxy; preferably R¹ is a phenyl substituted with one trifluoromethoxy, most preferably at the para position; preferably when A is carbon (C) R¹ is an unsubstituted phenyl; L² is a linking group, such that when A is nitrogen (N), L² is a group comprising 4-10 atoms (apart from hydrogen atoms), optionally forming a ring, whereof at least one of the atoms is nitrogen, the nitrogen forming part of an amide group; preferably the linking group is selected from a C₄₋₆-alkylamidylene and a pyrrolidinylene, said linking group optionally substituted with one or two of alkyl, hydroxy, oxo or thioxo group; most preferably L² is selected from butanamidylene, N-methylbutanamidylene, N,N-dimethylbutanamidylene, 4-hydroxybutanamidylene (HO—CH₂—C*H—CH₂—C(O)NH—, wherein the asterisk denotes attachment point), 4-oxobutanamidylene, 4-hydroxy-N-methylbutanamidylene, 4-oxo-N-methylbutanamidylene, 2-pyrrolidonyl, pyrrolidine-2,5-dionylene, 5-thioxo-2-pyrrolidinonylene and 5-methoxy-2-pyrrolidinonylene; and when A is carbon (C), then L² is either as defined for L² when A is nitrogen (N) or C₁₋₄ alkylene; L² is preferably methylene (—CH₂—); R² is a phenyl or a naphthyl, optionally substituted with halogen, preferably when R² is a phenyl it is substituted with halogen, preferably chlorine, at the para position, preferably when R² is unsubstituted naphthyl, L² is an alkylene group, preferably —CH₂—;

In some embodiments, the method comprises administering to a subject in need thereof at least one compound of general Formula (I) with a proviso that when A is carbon (C), L¹ is —NR⁴—, R⁴ is hydrogen, and R² is phenyl substituted with chlorine, then L² is not pyrrolidine-2,5-dione.

In some embodiments, R³ is hydrogen or heteroalkyl group comprising 3-12 atoms apart from hydrogen atoms, wherein at least one of said 3-12 atoms is a heteroatom, selected from nitrogen, sulfur and oxygen. In some embodiments, R³ is a C(O)NHCH₂C(O)OH group. In other embodiments (i.e., when A is nitrogen), R³ is absent.

In some embodiments, R⁴ is hydrogen.

In some embodiments, R1 is a phenyl substituted with trifluoromethoxy. In some embodiments, R¹ is a phenyl substituted with one trifluoromethoxy. In some embodiments, R¹ is a phenyl substituted with one trifluoromethoxy at the para position. In some embodiments, R¹ is phenyl.

In some embodiments, L² is a linking group, comprising 4-10 atoms (apart from hydrogen atoms), optionally forming a ring, whereof at least one of the atoms is nitrogen, said nitrogen forming part of an amide group; preferably said linking group is selected from a C₄₋₆-alkylamidylene and a pyrrolidinylene, the linking group optionally substituted with one or two of alkyl, hydroxy, oxo or thioxo group; most preferably L² is selected from butanamidylene, N-methylbutanamidylene, N,N-dimethylbutanamidylene, 4-hydroxybutanamidylene (HO—CH₂—C*H—CH₂—C(O)NH— wherein the asterisk denotes attachment point), 4-oxobutanamidylene, 4-hydroxy-N-methylbutanamidylene, 4-oxo-N-methylbutanamidylene, 2-pyrrolidonyl, pyrrolidine-2,5-dionylene, 5-thioxo-2-pyrrolidinonylene and 5-methoxy-2-pyrrolidinonylene. Each possibility represents a separate embodiment of the invention. In some embodiments, L² is 4-hydroxybutanamidylene (HO—CH₂—C*H—CH₂—C(O)NH—, wherein the asterisk denotes attachment point). In some embodiments, L² is C₁₋₄ alkylene, preferably methylene (—CH₂—).

The term “pyrrolidinylene” refers to a pyrrolidine ring as a bivalent substituent. Pyrrolidinylene include unsubstituted and substituted rings, such as, but not limited to, pyrrolidine-2-5-dione, 2-pyrrolidinone, 5-thioxo-2-pyrrolidinone, 5-methoxy-2-pyrrolidinone and the like.

In one embodiment, when A is nitrogen (N), the linking group L² is selected a C₄₋₆-alkylamidylene and a pyrrolidinylene, said linking group optionally substituted with one or two of alkyl, hydroxy, oxo or thioxo group. For example, L² may be butanamidylene, N-methylbutanamidylene, N,N-dimethylbutanamidylene, 4-hydroxybutanamidylene, 4-oxobutanamidylene, 4-hydroxy-N-methylbutanamidylene, 4-oxo-N-methyl-butanamidylene, 2-pyrrolidonyle, pyrrolidine-2,5-dionylene, 5-thioxo-2-pyrrolidinonylene or 5-methoxy-2-pyrrolidinonylene. Preferably, when L² is butanamidylene, N-methylbutanamidylene, N,N-dimethylbutanamidylene, 4-hydroxybutanamidylene, 4-oxobutanamidylene, 4-hydroxy-N-methylbutanamidylene or 4-oxo-N-methylbutanamidylene, then preferably the carbon in third position (C) of the butanamide moiety is bonded to the nitrogen (N) of the piperazine ring or the piperidine ring and the nitrogen (N) of the butanamide moiety is bonded to R². For example, when L² is 2-pyrrolidone, pyrrolidine-2,5-dione, 5-thioxo-2-pyrrolidone or 5-methoxy-2-pyrrolidone, then preferably a carbon (C) of the pyrrolidine moiety is bonded to the nitrogen (N) of the piperazine ring or the piperidine ring and the nitrogen (N) of the pyrrolidine moiety is bonded to R², in some embodiments. Alternatively, when L² is 4-hydroxybutanamidylene, then preferably a carbon (C) of the butanamidylene moiety is bonded to the nitrogen (N) of the piperazine ring and the nitrogen (N) of the butanamidylene moiety is bonded to R², in some embodiments.

In another embodiment, A is carbon (C), R³ is heteroalkyl and L² is methylene.

The invention also relates to the stereoisomers, enantiomers, mixtures thereof, and salts, particularly the physiologically acceptable salts, of the compounds of general Formula (I) according to the invention.

According to certain embodiments, the at least one piperazine- or piperidine-derivative is of general Formula Ia:

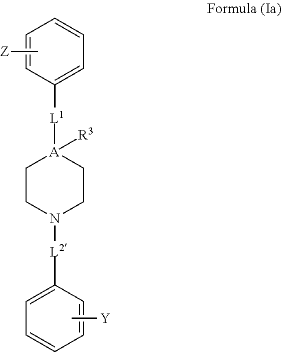

wherein: A, R³, Z and L¹ are as previously defined in reference to compound of Formula (I); preferably A is nitrogen (N); L²′ is a linking group selected from a C₄-alkylamidylene, a C₅-alkylamidylene and a C₆-alkylamidylene, optionally substituted with one or two of alkyl, hydroxy, oxo or thioxo group; preferably L²′ is selected from butanamidylene, N-methylbutanamidylene, N,N-dimethylbutanamidylene, 4-hydroxybutanamidylene, 4-oxobutanamidylene, 4-hydroxy-N-methylbutanamidylene or 4-oxo-N-methylbutanamidylene; most preferably L²′ is 4-hydroxybutanamidylene; wherein preferably the carbon (C) at position 3 of the alkyl moiety of alkylamidylene L²′ is bonded to the nitrogen (N) of the piperazine ring or of the piperidine ring, and the nitrogen (N) of the butanamide moiety is bonded to the phenyl group; preferably L^{2′} is HO—CH₂—C*H—CH₂—C(O)NH—, wherein the asterisk denotes attachment point; Y is halogen, preferably chlorine, e.g. at the para position; or an enantiomer, diastereomer, mixture or salt thereof.

According to certain embodiments, the piperazine- or piperidine-derivative is of general Formula (Ib):

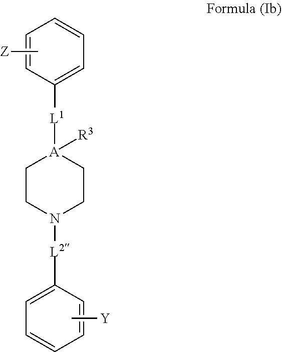

wherein: A, R³, and Z are as previously defined in reference to the compound of Formula (I); preferably A is nitrogen (N); L¹ is absent; L^{2″} is a pyrrolidinylene linking group, optionally substituted with one or two of alkyl, hydroxy, oxo or thioxo group, preferably L^{2″} is selected from 2-pyrrolidonylene, pyrrolidine-2,5-dionylene, 5-thioxo-2-pyrrolidinonylene and 5-methoxy-2-pyrrolidinonylene; most preferably L^{2″} is pyrrolidine-2,5-dionylene; wherein preferably a carbon (C) at position 4 or the carbon (C) at position 3 of the pyrrolidinyl moiety L^{2″} is bonded to the nitrogen (N) of the piperazine ring or the piperidine ring and the nitrogen (N) of the pyrrolidinyl moiety is bonded to the phenyl group substituted with Y; and Y is a halogen, preferably chlorine, e.g. at the para position.

According to certain embodiments, the piperazine- or piperidine-derivative is of general Formula (Ic):

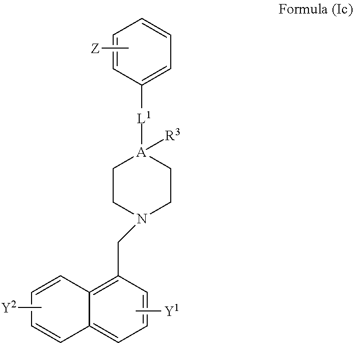

wherein: A, R³, and Z are as previously defined in reference to the compounds of general Formula (I); preferably wherein A is carbon (C); L¹ is —NH—; and Y¹ and Y² are each independently absent or a halogen; preferably wherein Y¹ and Y² are each independently absent; or an enantiomer, diastereomer, mixture or salt thereof. Preferred compounds of Formula (Ic) are those wherein R³ is —C(O)NHCH₂C(O)OH group, and/or wherein Z is C₁₋₂-alkoxy or halogenated C₁₋₂-alkoxy, e.g. C₁₋₂-perfluoroalkoxy.

According to certain embodiments, the piperazine- or piperadine-derivative is of general Formula (Id):

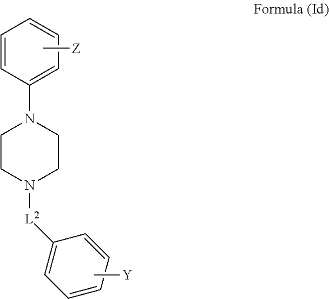

wherein: L² is selected from a C₄₋₆-alkylamidylene (e.g. HO—CH₂—C*H—CH₂—C(O)NH—, wherein the asterisk denotes attachment point), and a pyrrolidinylene (e.g. pyrrolidin-2,5-dionylene), optionally substituted with one or two of alkyl, hydroxy, oxo or thioxo group; and Z is haloalkoxy, e.g. C₁₋₂-perfluoroalkoxy, preferably, OCF₃, and Y is a halogen.

In some embodiments, L² is HO—CH₂—C*H—CH₂—C(O)NH—, wherein the asterisk denotes attachment point. In some embodiments, Z is OCF₃. In some embodiments, Y is chlorine. In some embodiments, Y is chlorine located para to L².

The invention also relates to the stereoisomers, enantiomers, mixtures thereof and salts thereof, of the compounds of general Formulae (Ia), (Ib), (Ic), and (Id), according to the invention. Table 1 provides non-limiting examples of compounds of general Formula (I). It includes the following compounds: N-(4-chlorophenyl)-4-hydroxy-3-(4-(4-(trifluoromethoxy)phenyl)-piperazin-1-yl)butanamide (Formula 1); 1-(4-chlorophenyl)-3-(4-(4-(trifluoromethoxy)phenyl)piperazin-1-yl)pyrrolidine-2,5-dione (Formula 2); (1-(naphthalen-2-ylmethyl)-4-(phenylamino)piperidine-4-carbonyl)glycine (Formula 3); 1-(4-chlorophenyl)-3-(4-(4-(trifluoromethoxy)phenyl)piperazin-1-yl)pyrrolidin-2-one (Formula 4); 1-(4-chlorophenyl)-5-thioxo-3-(4-(4-(trifluoro-methoxy)phenyl)piperazin-1-yl) pyrrolidin-2-one (Formula 5); 1-(4-chlorophenyl)-5-methoxy-4-(4-((4-(trifluoromethoxy)phenyl)amino) piperidin-1-yl)pyrrolidin-2-one (Formula 6); 1-(4-chlorophenyl)-5-thioxo-4-(4-((4-(trifluoromethoxy)phenyl)amino)piperidin-1 yl)pyrrolidin-2-one (Formula 7); 4-(4-chlorophenyl)-4-oxo-3-(4-(4-(trifluoromethoxy)phenyl)piperazin-1-yl)butanamide (Formula 8); and N-(4 chlorophenyl)-4-hydroxy-N-methyl-3-(4-(4-(trifluoro-methoxy)phenyl) piperazin-1-yl)butanamide (Formula 9).

TABLE 1
Examples of compounds of general Formula (I)

| Formula # | Structure | Description-according to general Formula (I) |
|---|---|---|
| 1 | 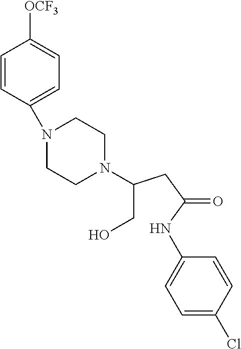 [SMILES: O=C(CC(CO)N1CCN(c2ccc(OC(F)(F)F)cc2)CC1)Nc1ccc(Cl)cc1] | A is nitrogen (N), R³ is absent, L¹ is absent, R¹ is phenyl substituted with one trifluoromethoxy located para to a nitrogen (N) of the piperazine ring, L² is 4-hydroxybutanamidylene, the 3^{rd} carbon (C) of the butanamide moiety is bonded to a nitrogen (N) of the piperazine ring, the nitrogen (N) of the butanamide moiety is bonded to R² and R² is a phenyl substituted with chlorine positioned para to the nitrogen (N) of the butanamide moiety [also identified herein as VBIT-4 or as BGD-4] |
| 2 | 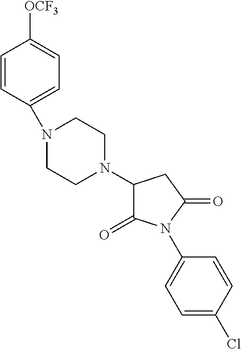 [SMILES: O=C1CC(N2CCN(c3ccc(OC(F)(F)F)cc3)CC2)C(=O)N1c1ccc(Cl)cc1] | A is nitrogen (N), R³ is absent, L¹ is absent, R¹ is phenyl substituted with one trifluoromethoxy located para to a nitrogen (N) of the piperazine ring, L² is pyrrolidine-2,5-dione, the carbon (C) at position 3 of the pyrrolidine-2,5-dione moiety is bonded to a nitrogen (N) of the piperazine ring, the nitrogen (N) of the pyrrolidine-2,5-dione moiety is bonded to R² and R² is a phenyl substituted with chlorine positioned para to the nitrogen (N) of the pyrrolidine-2,5-dione moiety [also identified herein as VBIT-3 or as BGD-3] |
| 3 | 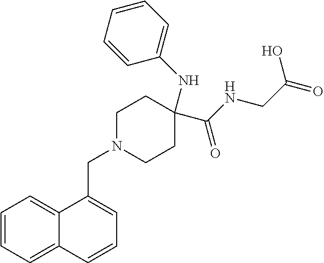 [SMILES: O=C(O)CNC(=O)C1(Nc2ccccc2)CCN(Cc2cccc3ccccc23)CC1] | A is carbon (C), R³ is —C(O)NHCH₂C(O)OH group; L¹ is —NH—, R¹ is a phenyl, L² is methylene and R² is a 1-naphthyl [also identified herein as VBIT-12] |
| 4 | 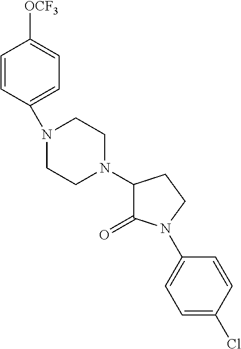 [SMILES: O=C1C(N2CCN(c3ccc(OC(F)(F)F)cc3)CC2)CCN1c1ccc(Cl)cc1] | A is nitrogen (N), R³ is absent, L¹ is absent, R¹ is a phenyl substituted with one trifluoromethoxy located para to a nitrogen (N) of the piperazine ring; L² is 2-pyrrolidone, the carbon (C) at position 3 of the pyrrolidone moiety is bonded to a nitrogen (N) of the piperazine ring, the nitrogen (N) of the pyrrolidone moiety is bonded to R² and R² is a phenyl substituted with chlorine positioned para to the nitrogen (N) of the pyrrolidone moiety [also identified herein as VBIT-5] |
| 5 | 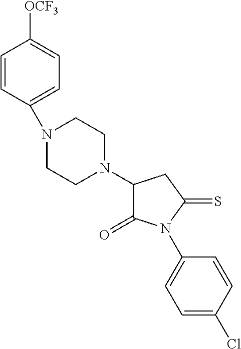 [SMILES: O=C1C(N2CCN(c3ccc(OC(F)(F)F)cc3)CC2)CC(=S)N1c1ccc(Cl)cc1] | A is nitrogen (N), R³ is absent, L¹ is absent, R¹ is a phenyl substituted with one trifluoromethoxy located para to a nitrogen (N) of the piperazine ring, L² is 5-thioxo-2-pyrrolidone, the carbon (C) at position 3 of the 5-thioxo-2-pyrrolidone moiety is bonded to a nitrogen (N) of the piperazine ring, the nitrogen (N) of the 5-thioxo-2-pyrrolidone moiety is bonded to R² and R² is a phenyl substituted with chlorine positioned para to the nitrogen (N) of the 5- thioxo-2-pyrrolidone moiety [also identified herein as VBIT-6] |
| 6 | 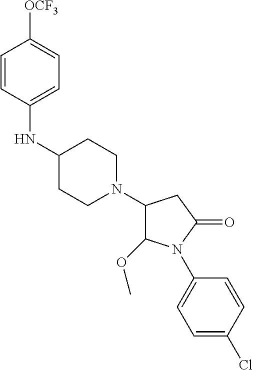 [SMILES: COC1C(N2CCC(Nc3ccc(OC(F)(F)F)cc3)CC2)CC(=O)N1c1ccc(Cl)cc1] | A is carbon (C), R³ is hydrogen, L¹ is —NH—, R¹ is a phenyl substituted with one trifluoromethoxy located para to L¹, L² is 5-methoxy-2-pyrrolidinone, the carbon (C) at position 3 of the 5-methoxy-2- pyrrolidinone moiety is bonded to the nitrogen (N) of the piperidine ring, the nitrogen (N) of the 5- methoxy-2-pyrrolidinone moiety is bonded to R² and R² is a phenyl substituted with chlorine positioned para to the nitrogen (N) of the 5- methoxy-2-pyrrolidinone moiety [also identified herein as VBIT-9] |
| 7 | 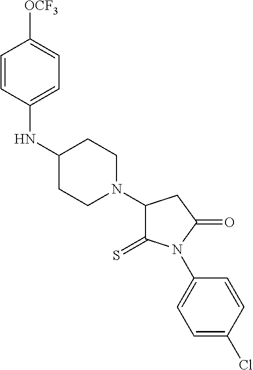 [SMILES: O=C1CC(N2CCC(Nc3ccc(OC(F)(F)F)cc3)CC2)C(=S)N1c1ccc(Cl)cc1] | A is carbon (C), R³ is hydrogen, L¹ is —NH—, R¹ is a phenyl substituted with one trifluoromethoxy located para to L¹, L² is 5-thioxo-2-pyrrolidone, the carbon (C) at position 3 of the 5-thioxo-2- pyrrolidone moiety is bonded to the nitrogen (N) of the piperidine ring, the nitrogen (N) of the 5-thioxo- 2-pyrrolidone moiety is bonded to R² and R² is a phenyl substituted with chlorine positioned para to the nitrogen (N) of the 5-thioxo-2-pyrrolidone moiety [also identified herein as VBIT-10] |
| 8 | 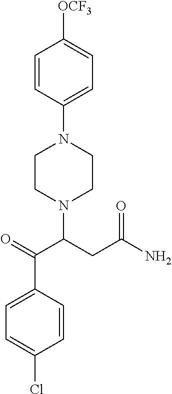 [SMILES: NC(=O)CC(C(=O)c1ccc(Cl)cc1)N1CCN(c2ccc(OC(F)(F)F)cc2)CC1] | A is nitrogen (N), R³ is absent, L¹ is absent, R¹ is phenyl substituted with one trifluoromethoxy located para to a nitrogen (N) of the piperazine ring, L² is 4-oxobutanamide, the 3^{rd} carbon (C) of the butanamide moiety is bonded to a nitrogen (N) of the piperazine ring, the 4^{th} carbon (C) of the butanamide moiety is bonded to R² and R² is a phenyl substituted with chlorine positioned para to the 4^{th} carbon (C) of the butanamide moiety [also identified herein as VBIT-7] |
| 9 | 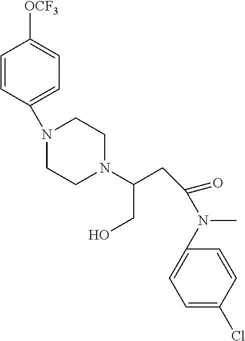 [SMILES: CN(C(=O)CC(CO)N1CCN(c2ccc(OC(F)(F)F)cc2)CC1)c1ccc(Cl)cc1] | A is nitrogen (N), R³ is absent, L¹ is absent, R¹ is phenyl substituted with one trifluoromethoxy located para to a nitrogen (N) of the piperazine ring, L² is 4-hydroxy-N-methylbutanamide, the 3^{rd} carbon (C) of the butanamide moiety is bonded to a nitrogen (N) of the piperazine ring, the nitrogen (N) of the butanamide moiety is bonded to R² and R² is a phenyl substituted with chlorine positioned para to the nitrogen (N) of the butanamide moiety [also identified herein as VBIT-8] |

In some embodiments, the piperazine- or piperidine derivative, also designated herein substituted N-heterocycle, is represented by a formula selected from Formula #1 (VBIT-4), Formula #2 (VBIT-3), Formula #3 (VBIT-12), Formula #4 (VBIT-5), Formula #5 (VBIT-6), Formula #6 (VBIT-9), Formula #7 (VBIT-10), Formula #8 (VBIT-7) or Formula #9 (VBIT-8) or enantiomers, diastereomers, mixtures or salts thereof. In some embodiments, the substituted N-heterocycle is selected from VBIT-4, VBIT-3, VBIT-12, VBIT-5, VBIT-6, VBIT-9, VBIT-10, VBIT-7 or VBIT-8 or enantiomers, diastereomers, mixtures or salts thereof. Each possibility represents a separate embodiment. In some embodiments, the substituted N-heterocycle is selected from VBIT-4, VBIT-3 or VBIT-12 or enantiomers, diastereomers, mixtures or salts thereof. In some embodiments, the substituted N-heterocycle is selected from VBIT-4 or VBIT-12 or enantiomers, diastereomers, mixtures or salts thereof. In some embodiments, the substituted N-heterocycle is selected from VBIT-4 or VBIT-3 or enantiomers, diastereomers, mixtures or salts thereof. In some embodiments, the substituted N-heterocycle is VBIT-4 or enantiomers, diastereomers, or salts thereof. In some embodiments, the substituted N-heterocycle is VBIT-12 or enantiomers, diastereomers, or salts thereof. In some embodiments, the substituted N-heterocycle is VBIT-3 or enantiomers, diastereomers, or salts thereof.

Some terms used herein to describe the compounds according to the invention are defined more specifically below.

The term “N-heterocycle”, and “nitrogen-heterocycle” are interchangeable and denote heterocyclic compounds having from 5 through 7 ring atoms, at least one of which is nitrogen. N-heterocycles encompass, inter alia, piperidine and piperazine.

The term “halogen” denotes an atom selected from among F, Cl, Br and I, preferably Cl and Br.

The term “heteroalkyl” as used herein in reference to R³ moiety of the general Formulae (I), (Ia), (Ib), (Ic), (Id), and (IIa), refers to a saturated or unsaturated group of 3-12 atoms (apart from hydrogen atoms), wherein one or more (preferably 1, 2 or 3) atoms are a nitrogen, oxygen, or sulfur atom, for example an alkyloxy group, as for example methoxy or ethoxy, or a methoxymethyl-, nitrile-, methylcarboxyalkylester- or 2,3-dioxyethyl-group; preferably heteroalkyl group is a chain comprising an alkylene, and at least one of a carboxylic acid moiety, a carbonyl moiety, an amine moiety, a hydroxyl moiety, an ester moiety, an amide moiety. The term heteroalkyl refers furthermore to a carboxylic acid or a group derived from a carboxylic acid as for example acyl, acyloxy, carboxyalkyl, carboxyalkylester, such as for example methylcarboxyalkylester, carboxyalkylamide, alkoxycarbonyl or alkoxycarbonyloxy; preferably the term refers to —C(O)NHCH₂C(O)OH group.

The term “C₁₋ₙ-alkyl”, wherein n may have a value as defined herein, denotes a saturated, branched or unbranched hydrocarbon group with 1 to n carbon (C) atoms. Examples of such groups include methyl, ethyl, n-propyl, iso-propyl, butyl, iso-butyl, sec-butyl, tert-butyl, n-pentyl, iso-pentyl, neo-pentyl, tert-pentyl, n-hexyl, iso-hexyl, etc.

The term “C₁₋₄-alkyl” denotes a saturated, branched or unbranched hydrocarbon group with 1 to 4 carbon (C) atoms.

The term “C₁₋ₙ-alkoxy”, wherein n may have a value as defined herein, denotes an alkyl group as defined herein, bonded via —O— (oxygen) linker.

The term “C₁₋ₙ alkylene”, wherein n may have a value as defined herein, denotes an alkylene group of saturated hydrocarbons substituents with the general formula CₙH₂ₙ. Generally, n is a positive integer. For example, Ci alkylene refers to methylene (—CH₂—), C₃ alkylene refers to C₃H₆, which may be n-propylene (—CH₂CH₂CH₂—) or isopropylene (—CH(CH₃)CH₂— or —CH₂CH(CH₃)—). Preferably the term refers to an unbranched n-alkylene.

The term “C₁₋ₙ-perfluoroalkoxy”, wherein n may have a value as defined herein, denotes an alkoxy group with hydrogen atoms substituted by fluorine atoms.

The term “C₁₋ₘ-alkylamidyl”, wherein m may have a value as defined herein, denotes a group comprising 1 to m carbon (C) atoms and an amide group formed by either Cₘ₋ₐalkyl-COOH and H₂N—Cₐalkyl, or Cₘ₋ₐalkyl-NH₂ and HOOC—Cₐalkyl, wherein a is smaller than or equal to m. Similarly, the terms C₄-alkylamidylene, C₅-alkylamidylene and C₆-alkylamidylene refer to divalent Cₘ-alkylamidyl groups, wherein m is either 4, 5, or 6, respectively.

Compounds of general Formulae (I), (Ia), (Ib), (Ic), and (Id) may be prepared according to methods known in the art (see, for example, WO 2018/116307 and US 2018/0078548, the content of which is incorporated by reference as if fully set forth herein).

The invention also relates to the stereoisomers, such as diastereomers and enantiomers, mixtures and salts, particularly the physiologically acceptable salts, of the compounds of general Formulae (I), (Ia), (Ib), (Ic), and (Id), and of the compounds of structural formulae 1, 2, 3, 4, 5, 6, 7, 8 and 9.

The compounds of general Formulae (I), (Ia), (Ib), (Ic), and (Id), or intermediate products in the synthesis of compounds of general Formulae (I), (Ia), (Ib), (Ic), and (Id), may be resolved into their enantiomers and/or diastereomers on the basis of their physical-chemical differences using methods known in the art. For example, cis/trans mixtures may be resolved into their cis and trans isomers by chromatography. For example, enantiomers may be separated by chromatography on chiral phases or by recrystallisation from an optically active solvent or by enantiomer-enriched seeding.

The compounds of general Formulae (I), (Ia), (Ib), (Ic), and (Id), and the compounds of structural formulae 1, 2, 3, 4, 5, 6, 7, 8 and 9, may be converted into the salts thereof, particularly physiologically acceptable salts for pharmaceutical use. Suitable salts of the compounds of general Formulae (I), (Ia), (Ib), (Ic), and (Id), and of the compounds of structural formulae 1, 2, 3, 4, 5, 6, 7, 8 and 9, may be formed with organic or inorganic acids including, but not limited to, hydrochloric acid, hydrobromic acid, sulfuric acid, phosphoric acid, lactic acid, acetic acid, succinic acid, citric acid, palmitic acid or maleic acid. Compounds of general Formulae (I), (Ia), (Ib), (Ic) and (Id), containing a carboxy group, may be converted into the salts thereof, particularly into physiologically acceptable salts for pharmaceutical use, with organic or inorganic bases. Suitable bases for this purpose include, for example, sodium hydroxide, potassium hydroxide, ammonium hydroxide, arginine or ethanolamine.

According to certain embodiments, the compound is of general Formula (IIa):

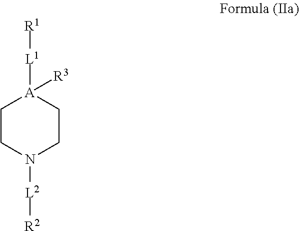

wherein: A is carbon (C); R³ is a hydrogen, an unsubstituted or substituted amide or a heteroalkyl group comprising 3-12 atoms apart from hydrogen atoms, wherein at least one of said 3-12 atoms is a heteroatom, selected from nitrogen, sulfur and oxygen; L¹ is an amino linking group —NR⁴—, wherein R⁴ is hydrogen, a C₁₋₅-alkyl, a C₁₋₅-alkylene or a substituted alkyl —CH₂R, wherein R is a functional group selected from hydrogen, halo, haloalkyl, cyano, nitro, hydroxyl, alkyl, alkenyl, aryl, alkoxyl, aryloxyl, aralkoxyl, alkylcarbamido, arylcarbamido, amino, alkylamino, arylamino, dialkylamino, diarylamino, arylalkylamino, aminocarbonyl, alkylaminocarbonyl, arylaminocarbonyl, alkylcarbonyloxy, arylcarbonyloxy, carboxyl, alkoxycarbonyl, aryloxycarbonyl, sulfo, alkylsulfonylamido, alkyl sulfonyl, aryl sulfonyl, alkylsulfinyl, arylsulfinyl or heteroaryl; when R³ is hydrogen, then L¹ is preferably —NH—; when R³ is heteroalkyl group comprising 3-12 atoms, then L¹ is preferably —NCₙH₂ₙ—, such that it forms a ring with R³; R¹ is an aromatic moiety, which is optionally substituted with one or more of C₁₋₂-alkoxy, e.g. haloalkoxy, such as C₁₋₂-perfluoroalkoxy; L² is a linking group comprising 4-10 atoms (apart from hydrogen atoms), optionally forming a ring, whereof at least one of the atoms is nitrogen, said nitrogen forming part of an amide group or L² is C₁₋₅ alkyl or C₁₋₅ alkylene; said linking group L² bonds piperidine or piperazine moiety at nitrogen (N) atom; preferably, L² is selected from butanamidylene, N-methylbutanamidylene, N,N-dimethylbutanamidylene, 4-hydroxybutanamidylene, 4-oxobutanamidylene, 4-hydroxy-N-methylbutanamidylene, 4-oxo-N-methylbutanamidylene, 2-pyrrolidonylene, pyrrolidine-2,5-dionylene, 5-thioxo-2-pyrrolidinonylene and 5-methoxy-2-pyrrolidinonylene; and R² is an aryl, optionally substituted with halogen, optionally when R² is a phenyl it is substituted with halogen, further optionally when R² is naphthyl, L² is an alkylenyl group. In a specific embodiment, R³ is hydrogen, L¹ is —NH—, and R¹ is a phenyl substituted with trifluoromethoxy. The invention also relates to use of the stereoisomers, enantiomers, mixtures thereof, and salts, particularly the physiologically acceptable salts, of the compounds of general Formula (I) and (IIa).
In some embodiments, A is carbon (C), R³ is hydrogen (H), L¹ is a NH group, R¹ is a phenyl substituted with one trifluoromethoxy, L² is pyrrolidine-2,5-dione, and R² is a phenyl substituted with a chlorine at the para position.

In some embodiments, A is carbon (C), R³ is a C(O)NCH₂C(O)OH group and is connected to both A and L¹, L¹ is a NCH₂ group and is connected to both 10 and R³, 10 is a phenyl, L² is methylene C¹ alkylene and R² is a naphthyl.

According to certain embodiments, methods of the present invention comprise administering to the subject at least one compound according to the general Formula (IIa), having a structural Formulae selected from Formula 10 and Formula 11:

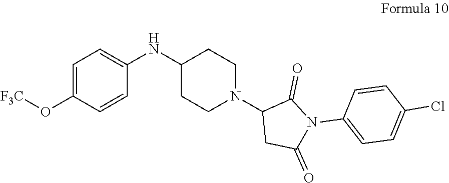

The compound of Formula 10 is also identified herein as AKOS022 or AKOS022075291.

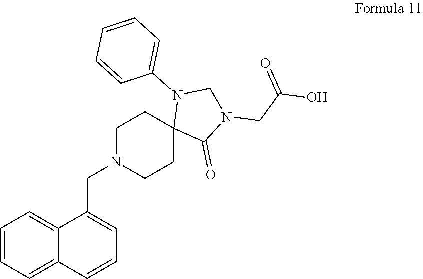 [SMILES: O=C(O)CN1CN(c2ccccc2)C2(CCN(Cc3cccc4ccccc34)CC2)C1=O]

The compound of Formula 11 is also identified herein as DIV 00781.

The compounds of general Formula (IIa) such as, without being limited to, the compounds of structural formulae 10 and 11, may be converted into the salts thereof, particularly physiologically acceptable salts for pharmaceutical use. Suitable salts of the compounds of general Formulae (IIa) include, but not limited to, the compounds of structural formulae 10 and 11, may be formed with organic or inorganic acids, such as, without being limited to hydrochloric acid, hydrobromic acid, sulfuric acid, phosphoric acid, lactic acid, acetic acid, succinic acid, citric acid, palmitic acid or maleic acid. Compounds of general Formula (IIa) containing a carboxy group, may be converted into the salts thereof, particularly into physiologically acceptable salts for pharmaceutical use, with organic or inorganic bases. Suitable bases for this purpose include, for example, sodium salts, potassium salts, arginine salts, ammonium salts, or ethanolamine salts.

### Peptides

The present invention is further based in part on the unexpected discovery that the N-terminus domain of VDAC1 is required for mtDNA interaction with VDAC1. The N-terminal domain contains three positively-charged residues (K12, R15, K20) that could interact with the negatively-charged backbone of mtDNA. Indeed, ISG expression was significantly reduced in mouse embryonic fibroblasts (MEFs) expressing either the VDAC1 mutated in the N-terminus or N-terminus truncated protein (ΔN-VDAC1), compared with those expressing WT VDAC1, indicating the importance of the N-terminal domain both in interacting with mtDNA and activating the cGAS pathway.

According to some embodiments, the VDAC inhibitor is a peptide derived from or corresponding to amino acids residues 1-26 of human VDAC1 N-terminal domain (SEQ ID NO:1) and comprising: (a) one or more mutations compared to the SEQ ID NO:1; (b) a truncation of at least 1 amino acid compared to SEQ ID NO:1; or any combination thereof, and wherein the mutated, truncated, or both, VDAC inhibiting peptide is devoid of pro-apoptotic activity.

In some embodiments, the VDAC inhibiting peptide does not induce, initiate, propagates, or any equivalent thereof, apoptosis. In some embodiments, the VDAC inhibiting peptide comprises at least 1 mutation wherein the mutation renders the peptide anti-apoptotic or non-pro-apoptotic.

It is to be understood that the present invention encompasses peptides having any length between 1-25 amino acids derived from or corresponding to amino acids residues 1-26 of human VDAC1 N-terminal domain, e.g., at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25 amino acids derived from or corresponding to amino acids residues 1-26 of human VDAC1 N-terminal domain.

According to certain embodiments, the peptide comprises 8 amino acids. According to other embodiments, the peptide comprises 12 amino acids. According to additional embodiments, the peptide comprises 16 amino acids. According to further embodiments, the peptide comprises 22 amino acids.

According to some embodiments, the VDAC inhibitor is a peptide of 1-25 amino acids comprising a contiguous sequence derived from amino acids residues 1-26 of human VDAC1 N-terminal domain. In some embodiments, the VDAC inhibiting peptide comprises less amino acids compared to SEQ ID NO:1. In some embodiments, the VDAC inhibiting peptide is a truncated form of SEQ ID NO:1. In some embodiments, the VDAC inhibiting peptide comprises one or more mutations and a truncation of at least 2 amino acids, compared to SEQ ID NO:1. In some embodiments, the VDAC inhibiting peptide comprises at least 2, at least 3, at least 4, or at least 5 mutations, or any value and range therebetween. Each possibility represents a separate embodiment of the invention. In some embodiments, the VDAC inhibiting peptide comprises 1-2, 1-3, 1-4, 1-5, 2-3, 2-4, 2-5, 3-4, 3-5, or 4-5 mutations, compared to SEQ ID NO:1. Each possibility represents a separate embodiment of the invention. In some embodiments, the mutation is located in the last 5 amino acids of the C′-terminal end of the VDAC inhibiting peptide. In some embodiments, the mutation is located in the GXXXG motif (SEQ ID NO:3) at the C-terminal end of the inhibiting peptide. In some embodiments, the truncation is an omission or deletion of at least 1, at least 2, at least 3, at least 4, or at least 5 amino acids, at the C′-terminal end of the VDAC inhibiting peptide, or any value and range therebetween. Each possibility represents a separate embodiment of the invention. In some embodiments, the truncation is an omission or deletion of 1-2, 1-3, 1-4, 1-5, 2-3, 2-4, 2-5, 3-4, 3-5, or 4-5 amino acids at the C′-terminal end of the VDAC inhibiting peptide. Each possibility represents a separate embodiment of the invention. In some embodiments the truncation is a complete or partial omission or deletion of the GXXXG motif (SEQ ID NO:3) at the C-terminal end of the inhibiting peptide. As used herein, “complete” is 100%, e.g., all 5 amino acids of the GXXXG motif are absent from the VDAC inhibiting peptide. As used herein, partially comprises 1-2, 1-3, 1-4, 2-3, 2-4, or 3-4 amino acids of the GXXXG motif are absent from the VDAC inhibiting peptide.

In some embodiments, the VDAC inhibiting peptide comprises or consists of the amino acid sequence: MAVPPTYADLGKSARDVFTKXYXFX (SEQ ID NO:2), wherein X is any amino acid other than glycine. In some embodiments, the VDAC inhibiting peptide comprises or consisting of an amino acid sequence selected from SEQ ID NO:4; SEQ ID NO:5; SEQ ID NO:6; SEQ ID NO:7; or SEQ ID NO:8.

In some embodiments, the VDAC inhibiting peptide comprises or consists of an amino acid sequence selected from SEQ ID:9; SEQ ID:10; SEQ ID:11; SEQ ID:12; or SEQ ID:13. In one embodiment the peptide of the invention comprises an amino acid sequence that modulates the interaction between VDAC1 and mtDNA. As used herein, the term “modulates” encompasses both “increase” and “increases”, or “decrease” and “decreases”.

It is yet another object of the present invention to provide short peptides based on the sequence of VDAC1 N-terminal domain and conjugates thereof comprising peptidomimetic compounds having further improved stability and cell permeability properties. Non-limiting examples of such compounds include N-alkylation of selected peptide residues, side-chain modifications of selected peptide residues, non-natural amino acids, use of carbamate, urea, sulfonamide and hydrazine for peptide bond replacement, and incorporation of non-peptide moieties including but not limited to piperidine, piperazine and pyrrolidine, through a peptide or non-peptide bond. Modified bonds between amino acid residues in peptidomimetics according to the present invention may be selected from: an amide, urea, carbamate, hydrazine or sulfonamide bond. Unless explicitly stated otherwise the bonds between the amino acid residues are all amide bonds.

Stability to enzymatic degradation is an important factor in designing a synthetic peptide to be used as a therapeutic agent. The D-stereoisomers of amino acids are known to be more stable to such degradation.

Thus, according to certain embodiments, the peptide of the invention is a L-stereomeric peptide, comprising only L-amino acids. According to other embodiments, the peptide is D-L stereomeric peptide, comprising a combination of D- and L-amino acids. According to yet additional embodiments, the peptide is D-stereomeric peptide, comprising only D-amino acids.

According to certain embodiments, the peptide based on the VDAC1 N-terminal domain is conjugated to a permeability-enhancing moiety covalently connected to the peptide via a direct bond or via a linker, to form a peptide conjugate.

The permeability-enhancing moiety according to the present invention may be connected to the C-terminus free group of the active peptide. The moiety may be linked directly to the peptide or through a linker or a spacer.

Any moiety known in the art to facilitate permeability actively or passively or enhance permeability of the compound into cells may be used for conjugation with the peptide core according to the present invention. Non-limiting examples include: hydrophobic moieties such as fatty acids, steroids and bulky aromatic or aliphatic compounds; moieties which may have cell-membrane receptors or carriers, such as steroids, vitamins and sugars, natural and non-natural amino acids, liposomes, nano-particles and transporter peptides. According to certain embodiments, the permeability-enhancing moiety is a cell penetrating peptide (CPP). In one exemplary embodiment the CPP is an amino acid sequence comprising the Drosophila antennapedia (ANTP) domain or a fragment thereof. In certain embodiments, the ANTP domain comprises the amino acid sequence as set forth in SEQ ID NO:14. According to these embodiments, the peptide conjugate comprises an amino acid sequence comprising SEQ ID NO:14 contiguously proceeded by any one of SEQ ID Nos.:4-13.

According to additional exemplary embodiments, the CPP comprises a fragment of the TIR domain recognized by the human transferrin receptor (Tf) having the amino acid sequence set forth in SEQ ID NO: 15 or SEQ ID NO:16. Each possibility represents a separate embodiment of the present invention. Other CPPs known in the art as TAT can also be used.

### Silencing Oligonucleotides

According to some embodiments, the VDAC inhibitor is a VDAC1-silencing oligonucleotide molecule, or a construct comprising same. Any VDAC1-silencing oligonucleotide molecule may be used in the methods of the present invention, as long as the oligonucleotide comprises at least 15 contiguous nucleic acids identical to SEQ ID NO:17, to an mRNA molecule encoded by same or to a sequence complementary thereto.

In some embodiments, the VDAC1-silencing oligonucleotide is at least 14 contiguous nucleic acids identical to SEQ ID NO:17, at least 15 contiguous nucleic acids identical to SEQ ID NO:17, at least 16 contiguous nucleic acids identical to SEQ ID NO:17, at least 17 contiguous nucleic acids identical to SEQ ID NO:17, at least 18 contiguous nucleic acids identical to SEQ ID NO:17, at least 19 contiguous nucleic acids identical to SEQ ID NO:17, at least 20 contiguous nucleic acids identical to SEQ ID NO:17, at least 21 contiguous nucleic acids identical to SEQ ID NO:17, at least 22 contiguous nucleic acids identical to SEQ ID NO:17, at least 23 contiguous nucleic acids identical to SEQ ID NO:17, at least 24 contiguous nucleic acids identical to SEQ ID NO:17, at least 25 contiguous nucleic acids identical to SEQ ID NO:17, at least 26 contiguous nucleic acids identical to SEQ ID NO:17, at least 27 contiguous nucleic acids identical to SEQ ID NO:17, at least 28 contiguous nucleic acids identical to SEQ ID NO:17, at least 29 contiguous nucleic acids identical to SEQ ID NO:17, or at least 30 contiguous nucleic acids identical to SEQ ID NO:17, or any value and range therebetween. Each possibility represents a separate embodiments of the invention. In some embodiments, the VDAC1-silencing oligonucleotide is 14 to 30 contiguous nucleic acids identical to SEQ ID NO:17, 15 to 28 contiguous nucleic acids identical to SEQ ID NO:17, 16 to 29 contiguous nucleic acids identical to SEQ ID NO:17, 22 to 26 contiguous nucleic acids identical to SEQ ID NO:17, 17 to 25 contiguous nucleic acids identical to SEQ ID NO:17, 16 to 24 contiguous nucleic acids identical to SEQ ID NO:17, 24 to 30 contiguous nucleic acids identical to SEQ ID NO:17, 16 to 23 contiguous nucleic acids identical to SEQ ID NO:17, or 18 to 26 contiguous nucleic acids identical to SEQ ID NO:17. Each possibility represents a separate embodiment of the invention.

According to certain embodiments, the VDAC1-silencing oligonucleotide comprises a nucleic acid sequence selected from SEQ ID NO:18; SEQ ID NO:19; SEQ ID NO:20; SEQ ID NO:21; SEQ ID NO:22; SEQ ID NO:23; SEQ ID NO:24; SEQ ID NO:25, SEQ ID NO:26, SEQ ID NO:27, SEQ ID NO:28, or a complementary sequence thereto.

According to certain embodiments, the VDAC1-silencing oligonucleotide is a RNA interference (RNAi) molecule or an antisense molecule. According to some embodiments, the RNAi molecule is an unmodified and/or modified double stranded (ds) RNA molecules including, but not limited to, short-temporal RNA (stRNA), small interfering RNA (siRNA), short-hairpin RNA (shRNA), and microRNA (miRNA).

According to certain exemplary embodiments, the RNAi is siRNA. According to some exemplary embodiments, the siRNA comprises a first oligonucleotide sequence identical to at least 15 nucleotides of SEQ ID NO:17 or to a mRNA encoded by same and a second oligonucleotide sequence substantially complementary to the first oligonucleotide; wherein said first and second oligonucleotide sequences are annealed to each other to form the siRNA molecule.

According to some embodiments, the siRNA is a single-stranded short hairpin RNA (shRNA) wherein the first oligonucleotide sequence is separated from the second oligonucleotide sequence by a linker which forms a loop structure upon annealing of the first and second oligonucleotide sequences. In some embodiments the linker is about 3 to about 60 nucleotides.

According to some exemplary embodiments, the siRNA comprises a first oligonucleotide having the nucleic acid sequence set forth in SEQ ID NO:18 and a second oligonucleotide having the nucleic acid sequence set forth in SEQ ID NO:31.

According to some exemplary embodiments, the siRNA comprises a first oligonucleotide having the nucleic acid sequence set forth in SEQ ID NO:19 and a second oligonucleotide having the nucleic acid sequence set forth in SEQ ID NO:26.

According to some exemplary embodiments, the siRNA comprises a first oligonucleotide having the nucleic acid sequence set forth in SEQ ID NO:20 and a second oligonucleotide having the nucleic acid sequence set forth in SEQ ID NO:27.

According to some exemplary embodiments, the siRNA comprises a first oligonucleotide having the nucleic acid sequence set forth in SEQ ID NO:25 and a second oligonucleotide having the nucleic acid sequence set forth in SEQ ID NO:28.

According to additional embodiments, at least one of the siRNA nucleic acids is chemically modified. Typically, the modification is 2′-O-methyl modification of a guanine or uracil. According to certain embodiments, the first and the second polynucleotide of the RNAi comprise several chemically modified guanine and/or uracil nucleotides.

According to certain exemplary embodiments, the modified siRNA molecule comprises a first oligonucleotide having the nucleic acid sequence set forth in SEQ ID NO:29 and a second oligonucleotide having the nucleic acid sequence set forth in SEQ ID NO:30.

According to certain exemplary embodiments, the modified siRNA molecule comprises a first oligonucleotide having the nucleic acid sequence set forth in SEQ ID NO:32, and a second oligonucleotide having the nucleic acid sequence set forth in SEQ ID NO:33.

According to certain embodiments, the method comprises administering to the subject a construct capable of expressing in cells of said subject a therapeutically effective amount of at least one VDAC1-silencing oligonucleotide. According to some embodiments, the method comprises administering to the subject a construct capable of expressing at least one oligonucleotide comprising a nucleic acid sequence selected from the group consisting of SEQ ID Nos:18-25. According to some embodiment, the method comprises administering to the subject a construct capable of expressing siRNA molecule comprising the nucleic acid sequence set forth in any one of SEQ ID Nos:26-28, and 31. According to certain exemplary embodiments, the method comprises administrating to the subject a construct capable of expressing siRNA oligonucleotide comprises a first oligonucleotide having the nucleic acid sequence set forth in SEQ ID NO:18 and a second oligonucleotide having the nucleic acid sequence set forth in SEQ ID NO:31. According to certain exemplary embodiments, the method comprises administrating to the subject a construct capable of expressing siRNA oligonucleotide comprises a first oligonucleotide having the nucleic acid sequence set forth in SEQ ID NO:29 and a second oligonucleotide having the nucleic acid sequence set forth in SEQ ID NO:30.

The silencing oligonucleotide molecules designed according to the teachings of the present invention can be generated according to any nucleic acid synthesis method known in the art, including both enzymatic syntheses and solid-phase syntheses. Any other means for such synthesis may also be employed; the actual synthesis of the nucleic acid agents is well within the capabilities of one skilled in the art and can be accomplished via established methodologies as detailed in, for example: Sambrook, J. and Russell, D. W. (2001), “Molecular Cloning: A Laboratory Manual”; Ausubel, R. M. et al., eds. (1994, 1989), “Current Protocols in Molecular Biology,” Volumes I-III, John Wiley & Sons, Baltimore, Md.; Perbal, B. (1988), “A Practical Guide to Molecular Cloning,” John Wiley & Sons, New York; and Gait, M. J., ed. (1984), “Oligonucleotide Synthesis”; utilizing solid-phase chemistry, e.g. cyanoethyl phosphoramidite followed by deprotection, desalting, and purification by, for example, an automated trityl-on method or HPLC.

It will be appreciated that nucleic acid agents of the present invention can be also generated using an expression vector as is further described herein below.

In some embodiments, the VDAC inhibiting compound reduces rates of mtDNA release from the mitochondria to the cytosol. In some embodiments, the VDAC inhibiting compound reduces the levels of mtDNA/fragments in the cytosol (e.g., cmtDNA). In some embodiments, the VDAC inhibiting compound maintains the levels of mtDNA/fragments in the mitochondria. In some embodiments, the VDAC inhibiting compound reduces the levels of VDAC oligomerization. In some embodiments, the VDAC inhibiting compound reduces the levels of VDAC mRNA. In some embodiments, the VDAC inhibiting compound reduces the stability of VDAC mRNA. In some embodiments, the VDAC inhibiting compound reduces the levels of the VDAC protein. In some embodiments, the VDAC inhibiting compound reduces the rates of VDAC protein synthesis. In some embodiments, the VDAC inhibiting compound reduces electrical conductance of the VDAC protein. In some embodiments, the VDAC inhibiting compound reduces the levels of type-1 interferon signaling. The terms “inhibit” and “reduce” are used herein interchangeably.

In some embodiments, the term “inhibit” refers to a reduction of at least 5%, at least 15%, at least 25%, at least 40%, at least 50%, at least 70%, at least 85%, at least 95%, at least 97, at least 99%, or 100% compared to control, or any value or range therebetween. In some embodiments, inhibit refers to a reduction of 5-15%, 10-25%, 20-40%, 30-50%, 45-70%, 65-85%, 80-95%, 90-97, 94-99%, or 95-100% compared to control. Each possibility represents a separate embodiment of the invention.

### Pharmaceutical Compositions

The present invention provides pharmaceutical compositions comprising one or more compounds of general Formulae (I), (Ia), (Ib), (Ic), (Id), and (IIa), such as, and without being limited to, the compounds of structural formulae 1, 2, 3, 4, 5, 6, 7, 8, 9, 10 and 11, particularly the specific compounds of Formulae 1, 2, 3, 10 and 11, or an enantiomer, diastereomer, mixture or a pharmaceutically acceptable salt thereof, and a pharmaceutically acceptable carrier or diluent, optionally further comprising one or more excipients, for use in treatment of a disease selected from an autoimmune disease and type-1 interferon-mediated diseases.

The present invention provides pharmaceutical compositions comprising the herein disclosed VDAC inhibiting peptide, and a pharmaceutically acceptable carrier or diluent, optionally further comprising one or more excipients, for use in treatment of a disease selected from type-1 interferon-mediated diseases, and an autoimmune disease.

The present invention provides pharmaceutical compositions comprising a VDAC1 silencing oligonucleotide, and a pharmaceutically acceptable carrier or diluent, optionally further comprising one or more excipients, or use in treatment of a disease selected from type-1 interferon-mediated diseases, and an autoimmune disease.

According to certain embodiments, the VDAC1-silencing oligonucleotide molecules of the present invention, particularly siRNA molecules are encapsulated in a particle suitable for the delivery of the siRNA to the site of action in a subject in need thereof. According to certain embodiments, the siRNA is encapsulated in a Poly(D, L-lactide-co-glycolide) (PLGA) based nanoparticle. According to certain embodiments, the PLGA-based nanoparticle further comprises polyethyleneimine (PEI), designated herein PEI-PLGA nanoparticle.

The present invention further provides a pharmaceutical composition comprising the unmodified and modified VDAC1-silencing oligonucleotides of the invention, a particle comprising same, and one or more pharmaceutically acceptable diluents, carriers or excipients.

According to certain embodiment, the composition is formulated for topical, intratumoral, intravenous or pulmonary administration.

The term “pharmaceutically acceptable” means approved by a regulatory agency of the Federal or a state government or listed in the U. S. Pharmacopeia or other generally recognized pharmacopeia for use in animals, and more particularly in humans.

The term “carrier” refers to a diluent, adjuvant, or vehicle with which the therapeutic compound is administered. Such pharmaceutical carriers can be sterile liquids, such as water and oils, including those of petroleum, animal, vegetable or synthetic origin, such as peanut oil, soybean oil, mineral oil, sesame oil and the like, polyethylene glycols, glycerin, propylene glycol or other synthetic solvents.

The compositions can take the form of solutions, suspensions, emulsion, tablets, pills, capsules, powders, patches, gels, creams, ointments, sustained-release formulations, and the like.

The pharmaceutical composition can further comprise pharmaceutical excipients including, but not limited to, wetting agents, emulsifying agents, and pH adjusting agents. Antibacterial agents such as benzyl alcohol or methyl parabens; antioxidants such as ascorbic acid or sodium bisulfite; chelating agents such as ethylenediaminetetraacetic acid; and agents for the adjustment of tonicity such as sodium chloride or dextrose are also envisioned.

For intravenous administration of a therapeutic compound, water is a preferred carrier. Saline solutions and aqueous dextrose and glycerol solutions can also be employed. Buffers can also be used.

Pharmaceutical compositions for parenteral administration can also be formulated as suspensions of the active compounds. Such suspensions may be prepared as oily injection suspensions or aqueous injection suspensions. For oily suspension injections, suitable lipophilic solvents or vehicles can be used including fatty oils such as sesame oil, or synthetic fatty acids esters such as ethyl oleate, triglycerides or liposomes. Aqueous injection suspensions may contain substances which increase the viscosity of the suspension, such as sodium carboxymethyl cellulose, sorbitol or dextran. Optionally, the suspension may also contain suitable stabilizers or agents which increase the solubility of the compounds, to allow for the preparation of highly concentrated solutions.

For transmucosal and transdermal administration, penetrants appropriate to the barrier to be permeated may be used in the formulation. Such penetrants, including for example DMSO or polyethylene glycol, are known in the art.

For oral administration, the compounds can be formulated readily by combining the active compounds with pharmaceutically acceptable carriers and excipients well known in the art. Such carriers enable the compounds of the invention to be formulated as tablets, pills, dragees, capsules, liquids, gels, syrups, slurries, suspensions, and the like, for oral ingestion by a subject. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose; and/or physiologically acceptable polymers such as polyvinylpyrrolidone (PVP). If desired, disintegrating agents may be added, such as cross-linked polyvinyl pyrrolidone, agar or alginic acid or a salt thereof such as sodium alginate.

In addition, enteric coating can be useful if it is desirable to prevent exposure of the compounds of the invention to the gastric environment.

Pharmaceutical compositions which can be used orally include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers.

In soft capsules, the active compounds may be dissolved or suspended in suitable liquids, such as fatty oils, liquid paraffin, or liquid polyethylene glycols. In addition, stabilizers may be added.

The compounds of general Formulae (I), (Ia), (Ib), (Ic), (Id), and (IIa), particularly of structural formulae 1, 2, 3, 4, 5, 6, 7, 8, 9, 10 and 11, more particularly the specific compounds of Formulae 1, 2, 3, 10 and 11, and the pharmaceutically acceptable salts thereof, may be formulated as nanoparticles. The nanoparticles may be prepared in well-known polymers, e.g. polylactic-co-glycolic acid. Generally, the compounds may be co-dissolved with the polymer in a suitable organic solvent, and the organic phase may be then dispersed in an aqueous phase comprising stabilizers and/or surface active agents. The stabilizer may be, e.g., polyvinyl alcohol. Upon evaporation of the organic solvent from the aqueous phase, the nanoparticles may be purified, e.g. by centrifugation and washing.

According to some embodiments, the pharmaceutical composition comprises a VDAC1-based peptide according to the present invention and a shielding particle. In certain embodiments the shielding particle comprises polyethyleneglycol (PEG) and/or lipids.

According to some embodiments, the VDAC1-silencing oligonucleotide molecule is encapsulated within Polyethylenimine (PEI)-Poly(D,L-lactide-co-glycolide) (PLGA) nanoparticle.

The composition can be formulated as a suppository, with traditional binders and carriers such as triglycerides, microcrystalline cellulose, gum tragacanth or gelatin.

Pharmaceutical compositions of the present invention may be manufactured by processes well known in the art, e.g., by means of conventional mixing, dissolving, granulating, grinding, pulverizing, dragee-making, levigating, emulsifying, encapsulating, entrapping or lyophilizing processes according to the general guidance provided in the art, e.g. by Remington, The Science and Practice of Pharmacy (formerly known as Remington's Pharmaceutical Sciences), ISBN 978-0-85711-062-6.

The dosage of a composition to be administered will depend on many factors including the subject being treated, the stage of the autoimmune disease, the route of administration, and the judgment of the prescribing physician.

The pharmaceutical compositions of the invention can further comprise one or more active agents known to treat an autoimmune disease, or one or more symptoms associated therewith.

### Therapeutic Use

The present invention provides methods for slowing the progression of or treating an autoimmune disease or one or more symptoms associated therewith comprising administering to a subject in need of such treatment a VDAC1 inhibiting compound or a pharmaceutical composition comprising same, thereby slowing the progression of or treating the autoimmune disease or one or more symptoms associated therewith.

The present invention provides methods for slowing the progression of or treating a NETosis-related autoimmune disease or one or more symptoms associated therewith comprising administering to a subject in need of such treatment a VDAC1 inhibiting compound or a pharmaceutical composition comprising same, thereby slowing the progression of or treating the NETosis-related autoimmune disease or one or more symptoms associated therewith.

The present invention provides methods for slowing the progression of or treating a type-1 interferon mediated disease or one or more symptoms associated therewith comprising administering to a subject in need of such treatment a VDAC1 inhibiting compound or a pharmaceutical composition comprising same, thereby slowing the progression of or treating the type-1 interferon mediated disease or one or more symptoms associated therewith.

In some embodiments, the method of the invention further comprises a step of selecting a subject suitable for treatment of a disease as disclosed herein. In some embodiments, a suitable subject has increased levels of: NET (i.e., NETosis), type-1 interferon signaling, cytosolic mtDNA, or any combination thereof, compared to control.

Methods for determining the levels of NETosis, type-1 interferon signaling and cytosolic mtDNA would be apparent to one of ordinary skill in the art, such as exemplified hereinbelow.

Non-limiting example for a protocol of quantification and visualization of NETs is as follows: NETs are induced in, for example normal-density granulocytes by incubating the cells with calcium ionophore A23187 (25 μM) in RPMI 1640 medium for 2 h, and NETs are then quantified using SYTOX fluorescent dye at 485/520 nm to quantify extracellular DNA. The fluorescence, of PicoGreen for example, at t=0 min is measured at 485/520 nm (emission/extinction) to quantify the total DNA. Another non-limiting example for NETs quantification includes fluorescence microscopy. Briefly, the cells are attached to coverslip chambers, stimulated for 90 min at 37° C. with calcium ionophore, fixed with 4% paraformaldehyde overnight at 4° C., and permeabilized with 0.2% Triton X-100 for 10 min, followed by 0.5% gelatin for 20 min. The cells are than stained with antibodies against human neutrophil elastase for 2 h at room temperature, washed in PBS, and stained with Hoechst 33342 and Alexa Fluor 488 secondary antibody for 2 h at room temperature. After mounting, the cells are visualized by confocal microscopy.

Without wishing to be bound by any theory or mechanism of action, the ability of the compounds of general Formulae (I), (Ia), (Ib), (Ic), (Id), and (IIa), particularly the compound having Formula 1 (VIBIT-4), to inhibit VDAC oligomerization, mtDNA release, type-1 interferon signaling, and neutrophil extracellular traps (NETs), contributes to their therapeutic effect in treating autoimmune diseases, e.g. SLE.

As used herein, the phrase “mtDNA leakage” encompasses the leakage of: intact mtDNA, leakage of mtDNA fragments, or a combination thereof.

In some embodiments, the method is directed to reducing or inhibiting the leakage of mtDNA, fragments thereof, or a combination thereof, from the mitochondria. In some embodiments, the method is directed to reducing the amount or level of circulating mtDNA. In some embodiments, the method is directed to inhibiting or reducing the amounts or levels of mtDNA/fragments in the matrix or intra-cristae space of the mitochondria, the peripheral space of the mitochondria, or both, the cytoplasm, the extracellular environment, the circulation (e.g., blood, serum), or any combination thereof. In some embodiments, autoimmune response, disease or disorder comprises mtDNA/fragment leakage.

As used herein, the term “intra-cristae space” refers to the space formed within the cristae of the mitochondrial inner membrane. As used herein, the term “peripheral space” refers to the space formed between the mitochondrial inner membrane and outer membrane.

Methods for determining the amount or level of mtDNA leakage or circulating mtDNA are common and would be apparent to one of ordinary skill in the art. A non-limiting example for a method of determining the amount of circulating mtDNA/fragments leakage, is exemplified hereinbelow, and includes but is not limited to real-time quantitative PCR and specific primers use.

In some embodiments, there is provided a composition for use in reducing the amount or level of circulating mtDNA/fragments, wherein circulating is in the cytoplasm, the extracellular environment, the circulation (e.g., blood, serum), or any combination thereof.

In some embodiments, the method is directed to treating an autoimmune disease or disorder by administering a therapeutically effective amount of VDAC inhibitor or a composition comprising thereof to a subject having increased circulating mtDNA/fragments amount or levels.

The term “VDAC” as used herein, unless the context explicitly dictates otherwise, refers to Voltage-Dependent Anion Channel proteins of a highly conserved family of mitochondrial porins. The term refers to all VDAC isoforms, e.g. to isoform VDAC1, to isoform VDAC2, or to isoform VDAC3.

The term “autoimmune disease” as used herein refers to a disorder resulting from an immune response against the subject's own tissue or tissue components or to antigens that are not intrinsically harmful to the subject. As used herein, the term autoimmune disease excludes Diabetes.

The symptoms and degree of severity can vary. Autoimmune diseases include, but are not limited to, autoimmune diseases that are frequently designated as involving single organ or single cell-type autoimmune disorder and autoimmune diseases that are frequently designated as involving systemic autoimmune disorder. Non-limiting examples of single organ or single cell-type autoimmune disorders include Hashimoto's thyroiditis, autoimmune hemolytic anemia, autoimmune atrophic gastritis of pernicious anemia, autoimmune encephalomyelitis, autoimmune orchitis, Goodpasture's disease, autoimmune thrombocytopenia, sympathetic ophthalmia, myasthenia gravis (MG), Graves' disease, primary biliary cirrhosis, chronic aggressive hepatitis, and membranous glomerulopathy. Non-limiting examples of autoimmune diseases involving systemic autoimmune disorder include systemic lupus erythematosis (SLE), rheumatoid arthritis (RA), multiple sclerosis (MS), Sjogren's syndrome, Reiter's syndrome, polymyositis-dermatomyositis, systemic sclerosis, polyarteritis nodosa, and bullous pemphigoid. Each possibility represents a separate embodiment of the invention.

According to one embodiment of the present invention, the autoimmune disease is systemic lupus erythematosus (SLE). According to another embodiment, the autoimmune disease is rheumatoid arthritis (RA). According to a further embodiment, the autoimmune disease is multiple sclerosis (MS), and the subject to be treated is mentally healthy, i.e., does not suffer from depression or any other mood disorder.

The term “NETosis-associated autoimmune disease” as used herein refers to any autoimmune disease or disorder which involves the release of neutrophil extracellular traps upon neutrophil cell death.

Alternatively or additionally, the autoimmune diseases that may be treated or prevented with the compositions of the present invention include those disorders involving tissue injury that occurs as a result of a humoral and/or cell-mediated response to immunogens or antigens of endogenous origin. Such diseases are frequently referred to as diseases involving the nonanaphylactic (i.e., Type II, Type III and/or Type IV) hypersensitivity reactions.

Type II hypersensitivity reactions (also referred to as cytotoxic, cytolytic complement-dependent or cell-stimulating hypersensitivity reactions) result when immunoglobulins react with antigenic components of cells or tissue, or with an antigen or hapten that has become intimately coupled to cells or tissue. Diseases that are commonly associated with Type II hypersensitivity reactions include, but are not limited, to autoimmune hemolytic anemia, erythroblastosis fetalis and Goodpasture's disease.

Type III hypersensitivity reactions, (also referred to as toxic complex, soluble complex, or immune complex hypersensitivity reactions) result from the deposition of soluble circulating antigen-immunoglobulin complexes in vessels or in tissues, with accompanying acute inflammatory reactions at the site of immune complex deposition. Non-limiting examples of prototypical Type III reaction diseases include systemic lupus erythematosis, rheumatoid arthritis, multiple sclerosis, serum sickness, certain types of glomerulonephritis, and bullous pemphingoid.

Type IV hypersensitivity reactions (frequently called cellular, cell-mediated, delayed, or tuberculin-type hypersensitivity reactions) are caused by sensitized T-lymphocytes which result from contact with a specific antigen. Non-limiting examples of diseases cited as involving Type IV reactions are contact dermatitis and allograft rejection.

The subject to be treated by the methods of the present invention is a human subject selected from the group consisting of a patient afflicted with the disease, a patient afflicted with the disease wherein the patient is in remission, a patient afflicted with the disease having manifested symptoms associated with the disease, and any combination thereof.

In some embodiments, the method of the invention further comprises a step of selecting a subject suitable for treatment using the VDAC inhibiting compound of the invention, wherein selecting comprises determining the subject has increased VDAC1 expression levels compared to healthy control.

In some embodiments, the method of the invention further comprises a step for monitoring the effectiveness or progression of treatment in the subject, wherein monitoring comprises determining the treated subject has reduced VDAC1 expression levels compared to a non-treated control. As used herein, non-treated control comprises an afflicted subject as disclosed hereinabove which was not administered with the VDAC inhibiting compound of the invention or an afflicted subject prior to treatment with the VDAC inhibiting compound of the invention.

In some embodiments, the method of the invention further comprises a step of selecting a subject suitable for treatment using the VDAC inhibiting compound of the invention, wherein selecting comprises determining the subject has increased NETosis compared to healthy control.

In some embodiments, the method of the invention further comprises a step for monitoring the effectiveness or progression of treatment in the subject, wherein monitoring comprises determining the treated subject has reduced NETosis compared to a non-treated control. As used herein, non-treated control comprises an afflicted subject as disclosed hereinabove which was not administered with the VDAC inhibiting compound of the invention or an afflicted subject prior to treatment with the VDAC inhibiting compound of the invention.

It will be appreciated by skilled artisans that many of the above-listed autoimmune diseases are associated with severe symptoms, the amelioration of which provides significant therapeutic benefit even in instances where the underlying autoimmune disease may not be ameliorated. The methods of the present invention find use in the treatment and/or prevention of myriad adverse symptoms associated with the above-listed autoimmune diseases.

As one specific example, systemic lupus erythematosis (SLE) is typically associated with symptoms such as fever, joint pain (arthralgias), arthritis, and serositis (pleurisy or pericarditis). In the context of SLE, the methods of the present invention are considered to provide therapeutic benefit when a reduction or amelioration of any of the symptoms commonly associated with SLE are achieved, regardless of whether the treatment results in a concomitant treatment of the underlying SLE.

In some embodiments, where the kidney function is compromised due to SLE (or other autoimmune disease), the treatment methods result in improvement of kidney function in the subject (e.g., slowing the loss thereof) as evaluated by, e.g., a change in proteinuria, albuminuria, etc.

Thus, in some embodiments, the methods of the present invention reduce the amount of protein secreted in the urine (proteinuria), amount of albumin secreted in the urine (albuminuria), and/or the patient's serum creatinine levels by at least 10%, 20%, 30%, 40%, 50%, 60%, 70%, or more, relative to control subjects. In other embodiments, the methods of the invention slow the loss of renal function by at least 10%, 20%, 30%, 40%, 50%, 60%, 70%, or more, relative to control subjects. Nonlimiting illustrative methods for assessing renal function are described in the Examples herein below.

As another example, rheumatoid arthritis (RA) typically results in swelling, pain, loss of motion and tenderness of target joints throughout the body. RA is characterized by chronically inflamed synovium that is densely crowded with lymphocytes. The synovial membrane, which is typically one cell layer thick, becomes intensely cellular and assumes a form similar to lymphoid tissue, including dentritic cells, T-, B- and NK cells, macrophages and clusters of plasma cells. This process, as well as a plethora of immunopathological mechanisms including the formation of antigen-immunoglobulin complexes, eventually result in destruction of the integrity of the joint, resulting in deformity, permanent loss of function and/or bone erosion at or near the joint. The methods may be used to treat or ameliorate anyone, several or all of these symptoms of RA. Thus, in the context of RA, the methods of the present invention are considered to provide therapeutic benefit when a reduction or amelioration of any of the symptoms commonly associated with RA is achieved, regardless of whether the treatment results in a concomitant treatment of the underlying RA and/or a reduction in the amount of circulating rheumatoid factor (“RF”).

As another specific example, multiple sclerosis (“MS”) cripples the patient by disturbing visual acuity; stimulating double vision; disturbing motor functions affecting walking and use of the hands; producing bladder incontinence; spasticity; and sensory deficits (touch, pain and temperature sensitivity). In the context of MS, the methods of the present invention are considered to provide therapeutic benefit when an improvement or a reduction in the progression of any one or more of the crippling effects commonly associated with MS is achieved, regardless of whether the treatment results in a concomitant treatment of the underlying MS. The methods of the present invention are aimed at treating subjects suffering from MS who do not suffer from depression or from any other mood disorder associated with MS.

The methods of the present invention are expected to slow the progression of an autoimmune disease, improve at least one symptom, and/or increase survival. For example, the methods of the present invention may result in a reduction in the levels of autoantibodies, B cells producing autoantibodies, and/or autoreactive T cells. The reduction in any of these parameters can be, for example, at least 10%, 20%, 30%, 50%, 70% or more as compared to pretreatment levels. Each possibility represents a separate embodiment of the present invention.

The term “therapeutically effective amount” as used herein with regard to a compound of the invention is an amount of a compound that, when administered to a subject will have the intended therapeutic effect, e.g. improving symptom(s) associated with an autoimmune disease. The full therapeutic effect does not necessarily occur by administering one dose, and may occur only after administering a series of doses. Thus, a therapeutically effective amount may be administered in one or more doses. The precise effective amount needed for a subject will depend upon, for example, the subject's weight, health and age, the nature of the autoimmune disease, the extent and severity of the symptoms of the specific autoimmune disease, the mode of administration of the pharmaceutical composition of the invention, and optionally, the combination of the pharmaceutical composition of the invention with additional active agent(s).

The term “treating” as used herein refers to inhibiting the disease state, i.e., arresting the development of the disease state or its clinical symptoms, or relieving the disease state, i.e., causing temporary or permanent regression of the disease state or its clinical symptoms. The term is interchangeable with any one or more of the following: abrogating, ameliorating, inhibiting, attenuating, blocking, suppressing, reducing, halting, alleviating or preventing the disease or any symptoms associated with the disease.

The term “preventing” as used herein means causing the clinical symptoms of the disease state not to develop in a subject that may be exposed to or predisposed to the disease state, but has not yet experienced or displayed symptoms of the disease state.

Animal models may serve as a resource for evaluating treatments for autoimmune diseases. For systemic lupus erythematosus (SLE), a mice model known as MRL-lpr is typically used. The MRL-lpr mice are homozygous for the lymphoproliferation spontaneous mutation (Fas^{lpr}) and show systemic autoimmunity, massive lymphadenopathy associated with proliferation of aberrant T cells, arthritis, and immune complex glomerulonephrosis. These mice are also useful as a model to therapies of Sjorgren (Sicca) syndrome. The well-established animal models of RA are: collagen type II induced arthritis in rats as well as in mice, adjuvant induced arthritis in rats, and antigen induced arthritis in several species. Each model represents a different mechanism underlying the disease expression. Several animal models are known for MS, three of which are mostly characterized: (1) the experimental autoimmune/allergic encephalomyelitis (EAE); (2) the virally-induced chronic demyelinating disease, known as Theiler's murine encephalomyelitis virus (TMEV) infection; and (3) the toxin-induced demyelination. All these models, in a complementary way, have allowed reaching a good knowledge of the pathogenesis of MS. Specifically, EAE is the model which better reflects the autoimmune pathogenesis of MS and is extremely useful to study potential experimental treatments. Experimental autoimmune encephalomyelitis (EAE) is an animal model of brain inflammation. It is an inflammatory demyelinating disease of the central nervous system (CNS). It is mostly used with rodents and is widely studied as an animal model of the human CNS demyelinating diseases, including multiple sclerosis and acute disseminated encephalomyelitis (ADEM). EAE is also the prototype for T-cell-mediated autoimmune disease in general. EAE can be induced in a number of species, including mice, rats, guinea pigs, rabbits and primates. The most commonly used antigens in rodents are spinal cord homogenate (SCH), purified myelin, myelin protein such as myelin basic protein (MBP), myelin proteolipid protein (PLP), and myelin oligodendrocyte glycoprotein (MOG), or peptides of these proteins, all resulting in distinct models with different disease characteristics regarding both immunology and pathology. It may also be induced by the passive transfer of T cells specifically reactive to these myelin antigens. Depending on the antigen used and the genetic make-up of the animal, rodents can display a monophasic bout of EAE, a relapsing-remitting form, or chronic EAE. The typical susceptible rodent will debut with clinical symptoms around two weeks after immunization and present with a relapsing-remitting disease. The archetypical first clinical symptom is weakness of tail tonus that progresses to paralysis of the tail, followed by a progression up the body to affect the hind limbs and finally the forelimbs. However, similar to MS, the disease symptoms reflect the anatomical location of the inflammatory lesions, and may also include emotional lability, sensory loss, optic neuritis, difficulties with coordination and balance (ataxia), and muscle weakness and spasms. Recovery from symptoms can be complete or partial and the time varies with symptoms and disease severity. Depending on the relapse-remission intervals, rats can have up to 3 bouts of disease within an experimental period.

The dose of the VDAC inhibiting compound required to achieve treatment of a disease usually depends on the pharmacokinetic and pharmacodynamic properties of the compound, which is to be administered, the patient, the nature of the disease, and the route of administration. Suitable dosage ranges for such compounds may be from 1.0 to 100 mg/kg body weight.

According to some embodiments, the methods of the present invention involve contacting a neutrophil with one or more compounds of the present invention, or a pharmaceutical composition comprising same in an amount effective to reduce mitochondrial DNA release and/or interferon gene expression and/or NETs formation by at least 10%, 20%, 30%, 40%, 50%, 60%, 70% or more as compared to pretreatment levels.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples. It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

### EXAMPLES

### Materials and Methods

### Cell Culture and Cellular Component Measurement

The following mouse embryonic fibroblasts (MEFs) were used: VDAC1/3^{-/-} (MEFs) and Bak/Bax^{-/-} MEFs with the respective WT counterparts; WT and cGAS^{-/-} MEFs; WT and IRF3/IRF7^{-/-} MEFs; and WT and MICU1^{-/-} MEFs. All MEFs were grown in complete Dulbecco's modified Eagle's medium (DMEM, Corning) supplemented with 10% fetal bovine serum (FBS, Sigma-Aldrich) and 1% penicillin and streptomycin (antibiotics, Gibco) at 37° C. with 5% CO₂. LMTK-1 cells were grown in complete RPMI 1640 medium (Gibco) supplemented with 10% FBS and antibiotics. The LMTK⁻-derived cell line lacking mtDNA (ρ^{o}), LMEB-4, was grown in LMTK-1 growth medium supplemented with uridine (50 μg/ml, Sigma-Aldrich) and sodium pyruvate (1 mM, Gibco). WT (ρ^{o}) and EndoG^{-/-} (ρ^{o}) MEFs were generated by incubation with ethidium bromide (Invitrogen) in complete DMEM supplemented with 15% FBS, antibiotics, uridine (50 μg/ml), and pyruvate (1 mM) for 5 months.

To generate MEFs with knockdown genes, MISSION shRNA Lentiviral Transduction Particles (Sigma-Aldrich) against mouse EndoG (SHCLNV-NM 007931) and TFAM (SHCLNV-NM_009360) were purchased from Sigma-Aldrich, and MEFs were transduced with the shRNA encoding lentivirus stocks in the presence of polybrene (8 μg/ml). Mouse cGAS (ON-TARGETplus Mb21d1 siRNA: 214763), STING (ON-TARGETplus Tmem173 siRNA: 72512), Tbk1 (ON-TARGETplus Tbk1 siRNA: 56480), ExoG (ON-TARGETplus ExoG siRNA: 208194), EndoG (ON-TARGETplus EndoG siRNA: 13804), and control siRNA (ON-TARGETplus Non-targeting siRNA) were purchased from Dharmacon. The siRNA transfection of MEFs was performed with 50 nM siRNA and Lipofectamine RNAiMAX reagent (Invitrogen) according to the manufacturer's instructions. To overexpress VDAC1 WT, VDAC1ΔN26, and VDAC1 with alanine mutation, a 26-aa truncated form of the VDAC N-terminus was subcloned or the WT VDAC1 gene was mutated using QuikChange Site-Directed Mutagenesis Kit (Stratagene) with the Primers: forward SEQ ID NO:34 and reverse SEQ ID NO:35. Transient DNA transfection into VDAC1/3^{-/-} MEFs was performed with Lipofectamine 3000 reagent (Invitrogen) according to the manufacturer's instructions.

Cellular cGAMP levels were measured by LC/MS (Agilent Technologies) using 5% perchloric acid extracts of WT and EndoG^{-/-} MEFs. Intracellular ROS production in MEFs was measured using the oxidative stress indicator CM-H₂DCFDA (Invitrogen) with flow cytometry (FACS). Mitochondrial ROS in MEFs was evaluated using the mitochondrial superoxide indicator MitoSOX (Invitrogen) with a confocal microscope (LSM880, Zeiss, and the fluorescence intensity was measured using Zen software (Zeiss). To quantify mitochondrial ROS in human PBMCs, the cells were obtained from heparinized blood using a Ficoll-Paque gradient. The cells were washed with PBS, resuspended in RPMI 1640 medium, and transferred to 96-well plates. Subsequently, the cells were stimulated with calcium ionophore A23187 (25 μM), VBIT-4 (5 μM), and MitoSOX (2 μM) (Life Technologies). After 1 h at 37° C., the fluorescence was measured at 510/595 nm using a microplate reader (Synergy HTX; BIOTEK). Cells without dye were used as the blank control.

### RNA Sequencing and Bioinformatic Analyses

Total cellular RNA was extracted from WT and EndoG^{-/-} MEFs using RNeasy Plus RNA extraction kits (QIAGEN) and the RNA sequencing was performed in the NIH-DNA Sequencing and Genomics core. RNA integrity was first verified by an Agilent Bioanalyzer. Starting from 500 ng of total RNA, TruSeq stranded total RNA library preparation kit (Illumina) was used to construct RNA-seq libraries following the manufacture's instruction. The resulting libraries were quantified by QuBit fluorometer (ThermoFisher) and sequenced on a Hiseq-3000 using a 2×50 bp modality. Data analysis of RNA sequencing results was performed in the NIH-Bioinformatics and Computational Biology Core Facility. Rigorous quality controls of paired-end reads were assessed using FastQC tools. Gene expression levels were estimated for the GENCODE GTF reference database. Cohort gene expression data was then assessed for outliers and irregular characteristics by reviewing properties of summary distributions by unsupervised principle component analysis (PCA) using R and manual review of the outcome. Differential expression analysis at the gene-level was carried out using limma open source R/Bioconductor packages. The lmFit function in limma was used to Fit linear models for each gene to calculate log 2 fold changes and p-values using the normalized factors as weights in the model. To account for multiple testing, the false discovery rate (FDR) via the Benjamani-Hochberg algorithm was calculated. The inventors then used the R statistical software environment using the GSEA and GAGE Bioconductor packages to carry out the gene set enrichment analyses on pre-defined gene ontology (GO) gene sets. The GO categories included were Biological Process (BP), Cellular Component (CC) and Molecular Function (MF). FDR q-values were estimated to correct the p-values for the multiple testing issue.

### RNA Extraction and Real-Time PCR

Total RNA was extracted using RNeasy Plus Mini Kit (Qiagen) according to the manufacturer's instructions. The first-strand cDNA was synthesized from 2 μg purified mRNA using Accupower RT PreMix (BioNeer). The reaction mixtures were incubated at 42° C. for 60 min and 94° C. for 5 min. Real-time RT-PCR was performed using the LightCycler 96 system (Roche Life Science) with SYBR Green master mix (Roche). The primers used were as follow (5′-3′): EndoG forward (SEQ ID NO:36), and reverse (SEQ ID NO:37); Cxcl10 forward (SEQ ID NO:38), and reverse (SEQ ID NO:39); GAPDH forward (SEQ ID NO:40), and reverse (SEQ ID NO:41); Ifi44 forward (SEQ ID NO:42), and reverse (SEQ ID NO:43); Ifit1 forward (SEQ ID NO:44), and reverse (SEQ ID NO:45); Ifit3 forward (SEQ ID NO:46), and reverse (SEQ ID NO:47); IFNα4 forward (SEQ ID NO:48), and reverse (SEQ ID NO:49); IFNβ forward (SEQ ID NO:50), and reverse (SEQ ID NO:51); Iigp1 forward (SEQ ID NO:52), and reverse (SEQ ID NO:53); ISG15 forward (SEQ ID NO:54), and reverse (SEQ ID NO:55); Oasl2 forward (SEQ ID NO:56), and reverse (SEQ ID NO:57); Rsad2 forward (SEQ ID NO:58), and reverse (SEQ ID NO:59); USP18 forward (SEQ ID NO:60), and reverse (SEQ ID NO:61); VDAC1 forward (SEQ ID NO:62), and reverse (SEQ ID NO:63). GAPDH was used as an internal standard of mRNA expression, and the ratio of the target gene expression to GAPDH expression was calculated using LightCycler 96 Instrument software (Roche). The quality of real-time RT-PCR results was evaluated based on the melting temperature (Tₘ) of a DNA fragment and melting curve analysis.

### Cell Lysate Preparation and Western Blot Analysis

MEFs were harvested and washed twice with ice-cold PBS, and the pellets were lysed on ice for 30 min in RIPA buffer (50 mM Tris-HCl pH 7.4, 0.15 M NaCl, 1.0 mM EDTA, 1% NP-40, 0.25% sodium deoxycholate) freshly supplemented with phosphatase and protease inhibitors (Roche). Nuclear extracts were obtained using NE-PER Nuclear and Cytoplasmic Kit (Pierce) according to the manufacturer's instructions. The total protein concentration was determined by Coomassie Plus protein assay (Thermo Scientific) and subjected to western blotting. The following antibodies were used: ISG15 (#2743, Cell Signaling); EndoG (ab76122, abcam); VDAC1 (ab14734, abcam); phospho-IRF3 (#29047, Cell Signaling); IRF3 (#4302, Cell Signaling); phospho-TBK (#5483, Cell Signaling); p-STAT1 (#9167, Cell Signaling); lamin B1 (#13435, Cell Signaling); P62 (#5114, Cell Signaling); LC3A/B (#4108, Cell Signaling); IFI44 (MBS2528890, MyBioSource); α-tubulin (sc-8035, Santa Cruz).

Quantification of mtDNA Release

MEFs were resuspended in 170 μl digitonin buffer containing 150 mM NaCl, 50 mM HEPES pH 7.4, and 25 μg/ml digitonin (EMD Millipore Corp). The homogenates were incubated on a rotator for 10 min at room temperature, followed by centrifugation at 16,000 g for 25 min at 4° C. A 1:20 dilution of the supernatant (cmtDNA) was used for real-time RT-PCR. The pellet was resuspended in 340 μl lysis buffer containing 5 mM EDTA and proteinase K (Qiagen) and incubated at 55° C. overnight. The digested pellet was diluted with water (1:20 to 1:100) and heated at 95° C. for 20 min to inactivate proteinase K, and the sample was used for real-time PCR. The primers used were as follow (5′-3′): D-loop1 forward (SEQ ID NO:64), and reverse (SEQ ID NO:65); D-loop2 forward (SEQ ID NO:66), and reverse (SEQ ID NO:67); D-loop3 forward (SEQ ID NO:68), and reverse (SEQ ID NO:69). The cmtDNA in the supernatant was normalized to the total mitochondrial DNA in the pellet for each sample.

Mitoplasts were isolated from the mitochondria of mouse liver. The liver tissue was washed twice with ice-cold PBS and minced in mitochondrial isolation buffer containing 225 mM mannitol, 75 mM sucrose, 5 mM MOPS, 0.5 mM EGTA, and 2 mM taurine (pH 7.25) with a protease inhibitor cocktail (Roche). The cells were ruptured by 10 Dounce homogenizer strokes using pestle A (large clearance) for the initial strokes, followed by pestle B using a pre-chilled Dounce homogenizer (Abcam) for 25 strokes. The homogenized samples were centrifuged at 1000 g for 10 min at 4° C. The supernatant was transferred to a new tube and centrifuged at 1,000 g for 5 min at 4° C., and the mitochondrial pellet was collected after the centrifugation of the final supernatant at 11,500 g for 10 min at 4° C. Mitochondria were incubated in 20 mM KH₂PO₄ buffer for 40 min in a cold room. After gentle agitation with a pipette, the samples were centrifuged at 4° C. for 10 min at 8000 g. The mitoplasts were resuspended in mitoplast swelling buffer containing 125 mM sucrose, 50 mM KCl, 5 mM HEPES, 2 mM KH₂PO₄, and 1 mM MgCl₂ (pH 7.2). The swelling reactions were energized with 20 mM succinate to support swelling using 0.1 mM H₂O₂, 600 μM Fe²⁺, and 250 μM Ca²⁺ for 10 min at room temperature with or without pre-incubation with 1.6 μM CysA. The reaction was carried out with 100 μg mitoplast protein in 200 μl solution at 28° C. for 10 min. In addition, 2 mM EDTA was added to prevent DNA degradation in the samples. After centrifugation at 21,000 g for 15 min at 4° C., the mtDNA in the supernatant was purified using QIAamp DNA Micro Kit (Qiagen), and the mtDNA was detected using mouse mtDNA-specific D-loop3 primer.

For the quantification of fimtDNA release, 143B cells were resuspended in mitochondrial isolation buffer and subsequently homogenized 30 times with pestle B (small clearance). The homogenized samples were centrifuged at 1,000 g for 10 min at 4° C. The supernatant was transferred to a new tube and centrifuged at 1,000 g for 5 min at 4° C., and the mitochondrial pellet was collected after the centrifugation of the final supernatant at 11,500 g for 10 min at 4° C. Isolated mitochondria were resuspended in 50 μl CSK buffer containing 10 mM PIPES pH 6.8, 300 mM sucrose, 100 mM NaCl, 3 mM MgCl₂, 1 mM EGTA, and 0.05% Triton X-100, for 5 min on ice, and the supernatant (fimtDNA) and CSK-pellet fractions were collected after centrifugation at 17,000 g for 30 min at 4° C. A 1:20 dilution of CSK-sup was used for real-time PCR with fimtDNA primers in each reaction. The primers used were as follow (5′-3′): hfimtNDA forward (SEQ ID NO:70), and reverse (SEQ ID NO:71). CSK-pellet was resuspended in 100 μl lysis buffer containing 20 mM EDTA and proteinase K and incubated at 56° C. overnight. The digested pellet was diluted with water (1:20 to 1:100) and heated at 95° C. for 20 min to inactivate proteinase K, and the CSK-pellet fraction was used for real-time PCR in each reaction. The fimtDNA in CSK-sup was normalized to the mtDNA in CSK-pellet.

Sequencing of fimtDNA and cmtDNA

To prepare the fimtDNA for the sequencing, we isolated the pure mitochondria from MEFs without contamination from other organelle using percoll gradient method. The fmtDNA was prepared by incubating the pure mitochondria in the CSK buffer for 5 min on ice, and cmtDNA was prepared by incubating the MEFs in the digitonin buffer for 10 min. Purified fimtDNA and cmtDNA were used to construct NextGen sequencing libraries with ThruPLEX Plasma-seq Kit (Takara) following the manufacturer's instructions. Sequencing data were acquired using the Illumina MiSeq platform with a 2×75 bp modality. Raw sequence reads were first mapped to the GRCm38 mouse reference genome excluding the mitochondrial genome reference by Burrows-Wheeler Aligner (BWA) software (version 0.7.17) with default settings. In addition, the unmapped reads were saved and aligned to the GRCm38 mitochondrial genome by BWA with default settings. The SAMtools software (version 1.6) provided statistical information on the coverage of mtDNA and the insert size of the paired mapped reads. The insert size distribution was computed and plotted by the Kernel density estimation function in the R stat package.

### HSV-1 Infection and HSV-1-RFP Growth Curve

HSV-1 encoding mRFP1 fused to the N-terminus of VP26 (clone HSV F-GS 2822) was used. The virus was titrated in both Vero cells and WT MEFs. To determine the plaque and infected cell morphology, EndoG^{-/-} MEFs and VDAC1/3^{-/-} MEFs with the respective WT counterparts were maintained in DMEM supplemented with 15% FBS, 1% Penicillin-Streptomycin-Glutamine, and 1 mM sodium pyruvate. MEFs were seeded in 12-well cell culture plates so that they will be 100% confluent at infection. HSV-1-RFP was added and incubated at 37° C. with 5% CO₂ for 1-2 days until isolated plaques were formed. The plaque size and red fluorescence intensity were determined, and micrographs were taken using a UV fluorescence microscope (Olympus IX51). To determine the percentage of MEFs infected with HSV-1-RFP by FACS, MEFs were seeded in 12-well plates so that the monolayers will be 100% confluent at infection and incubated with HSV-1-RFP at 37° C. with 5% CO₂ overnight. Then, the MEFs were dissociated with TrypLE Select (Gibco) to form a single cell suspension, fixed with 4% PBS-buffered formaldehyde on ice, washed with PBS, and resuspended in 0.2 ml PBS containing 2% FBS and 1 mM EDTA. FACS was performed to determine the percentage of HSV-1-RFP-positive MEFs. To determine the HSV-1-RFP growth curve, EndoG^{-/-} MEFs and VDAC1/3^{-/-} MEFs with the respective WT counterparts were seeded in 12-well plates 1 day before infection and infected with HSV-1-RFP. Two aliquots of the input virus were stored as 0 h samples, and infected cell plates were incubated at 37° C. with 5% CO₂ for 1 h. The inoculum was removed from the 12-well plates, and 1 ml of growth medium was added. At 3, 9, and 24 h post-infection, the infected MEFs were scraped in their culture supernatant and subjected to 3 cycles of freeze and thaw, followed by centrifugation to remove cell debris. The cell-free virus in the supernatant was stored at −80° C. in a freezer and subsequently titrated in Vero cells.

### Mitochondrial and Recombinant VDAC1 Purification, Channel Reconstitution, Recording and Analysis

VDAC1 protein was purified from rat liver mitochondria using celite: hydroxyapatite CMC chromatography method as previously described (Ben-Hail and Shoshan-Barmatz (2014)). Vectors expressing full length murine mVDAC1 and N-terminal (1-26) truncated mVDAC1 (ΔN-VDAC1) were cloned into pcDNA4/TO vector (Invitrogen) as described previously (Abu-Hamad et al., 2009). HEK-293 cells silenced for human hVDAC1 expression were transfected with pcDNA3.1 plasmid encoding either mVDAC1 or ΔN-mVDAC1, using Jet-Prim. Cells were harvested 48 h post-transfections, and the proteins were purified as above for mitochondrial VDAC1 (Ben-Hail and Shoshan-Barmatz (2014). The reconstitution of mitochondria purified VDAC1 or recombinant WT or ΔN-VDAC1 into a planar lipid bilayer (PLB) and subsequent single and multiple channel current recordings and data analysis were carried out (Ben-Hail and Shoshan-Barmatz (2014)). Briefly, the PLB was prepared from soybean asolectin dissolved in n-decane (30 mg/ml). Purified VDAC1 was added to the chamber defined as the cis side containing 1 M NaCl, 10 mM Hepes, pH 7.4. Currents were recorded before and 15 minutes after the addition of 37 nM mtDNA (47 bp) in the cis or trans compartment, under voltage-clamp using a Bilayer Clamp BC-535B amplifier (Warner Instrument, Hamden, Conn.). The currents, measured with respect to the trans side of the membrane (ground), were low-pass-filtered at 1 kHz and digitized online using a Digidata1440-interface board and pClampex 10.2 software (Axon Instruments, Union City, Calif.).

mtDNA Efflux from Liposomes Reconstituted with VDAC1

Liposomes were prepared by the extrusion method using mini-extruder purchased from Avanti Polar Lipids Inc. (Alabaster, Ala.). Briefly, a thin lipid film was obtained by dissolving soybean asolectin (10 mg/ml of chloroform) and then evaporating chloroform slowly under a gentle stream of nitrogen gas. Then, lipid film was hydrated in a buffer (10 mM Tricine, 150 mM NaCl, pH 7.4) containing 100 nM of mtDNA (47 bp) for 30-60 min at room temperature with 5 vortex cycles (1 minute separated by 1 minute rest). Then, mtDNA was added to the suspension of large multilamellar vesicles, exposed to five freeze-thaw cycles using liquid nitrogen and passed 11 times through the mini-extruder containing a polycarbonate filter (Whatman) to get the mtDNA loaded-liposomes. mtDNA loaded-liposomes were equally divided into two aliquots for making VDAC1-containing and VDAC1-free liposomes. Incorporation of purified VDAC1 (30 μg/ml) into the mtDNA-loaded liposomes solution was performed by incubating the liposomes with VDAC1 for 20 min at RT, followed by three freeze-thaw cycles and mild sonication. VDAC1-free liposomes were similarly prepared by using VDAC1-column elution buffer instead of VDAC1. Samples were centrifuged for 15 min at 100,000 g and pellets were re-suspended in buffer (10 mM Tricine, 150 mM NaCl, pH 7.4). Liposomes were diluted 4-fold and 40 min later were centrifuged for 15 min at 100,000 g and supernatant aliquot were analyzed for mtDNA using qPCR with mtDNA specific primer of the D-loop3 region.

### Mitochondrial Swelling Assay and Ca²⁺ Accumulation Analysis

The PTP opening was analyzed following mitochondria swelling. Briefly, freshly isolated mitochondria (0.5 mg/ml) were incubated for 2 min at 24° C. with the indicated concentrations of VBIT-4 for Ca²⁺-induced mitochondrial swelling assay. Swelling was initiated by the addition of Ca²⁺ (0.1 mM) to the sample cuvette. Absorbance changes at 520 nm were monitored every 16 s for 15 min. Cyclosporine A 10 μM) was used as a positive control. Results are shown as a percentage of control. Ca²⁺ accumulation by freshly isolated rat liver mitochondria (0.5 mg/ml) was assayed with the indicated concentrations of VBIT-4 in the presence of 120 mM CaCl₂) (containing [⁴⁵Ca²⁺]), 220 mM mannitol, 70 mM sucrose, 5 mM succinate, 0.15 mM Pi and 15 mM Tris/HCl, pH 7.2. Ca²⁺ uptake was terminated by rapid Millipore filtration (0.45 μm).

### VDAC1 Cross-Linking Assay

Purified VDAC1 (16 μg/ml) was incubated with 60 nM of mtDNA (120 bp) for 15 min at 25° C. in 20 mM Tricine, pH 8.4 and then incubated for 15 min at 30° C. with the cross-linking reagent EGS (100 μM). Samples (0.1-1 μg protein) were subjected to SDS-PAGE and immunoblotting using anti-VDAC1 antibodies. Quantitative analysis of immuno-reactive VDAC1 dimer, trimer and multimer bands was performed using FUSION-FX (Vilber Lourmat, France).

mtDNA-Peptide Binding Assay

C-terminal biotinylated peptides corresponding to amino acid residues from 1 to 26 of mouse VDAC1 (SEQ ID NO:1) and a mutant peptide (SEQ ID NO:72) with acetylation (N-terminus) and amidation (C-terminus) were synthesized and purified by Genscript (Piscataway, N.J., USA). Mitochondrial DNA was amplified using PCR with mtDNA specific primer of the D-loop region. The primers used were as follow (5′-3′): mtDNA 120 bp forward (SEQ ID NO:73), and reverse (SEQ ID NO:74). Purified mtDNA (120 bp) was incubated rotating end-over-end with peptides and Streptavidin Dynabeads (Invitrogen) for 18 h at 4° C. The peptides were captured by the Streptavidin Dynabeads, and unbound peptides and free mtDNA were removed by extensive washing with PBS. The samples were treated with proteinase K for 30 min at 60° C., and the mtDNA in the supernatant was purified with QIAquick Nucleotide Removal Kit (Qiagen). The purified mtDNA was quantified using real-time RT-PCR with the D-loop3 primers.

### Lupus Animal Model

All experiments were approved by the ACUC (Animal Care and Use Committee) of the NIH/NHLBI. Lupus-prone female MRL/MpJ-Fas^{lpr}/J mice (stock #000485, n=10 in each group), used as a model to determine the etiology of systemic lupus erythematosus (SLE), were purchased from The Jackson Laboratory. VIBIT4 was freshly dissolved in DMSO and diluted in water (final pH 5.0). The mice were treated with a daily dose of VBIT-4 (20 mg/kg) or vehicle in drinking water for 5 weeks, beginning at 11 weeks of age until euthanasia at 16 weeks of age. Blood and urine samples were collected when the mice were 16 weeks of age. The body weight of the mice was measured before and after VBIT-4 administration (at 11 and 16 weeks of age).

### Albumin:Creatinine Ratio in the Urine

Proteinuria in fresh urine was measured using creatinine and albumin ELISA kits (Exocell), and mouse albumin was used to determine the proteinuria:creatinine ratio following the manufacturer's instructions.

Quantification of mtDNA and Anti-dsDNA Antibodies in Lupus Serum

Circulating mtDNA was isolated from 500 μl of serum using QIAamp Circulating Nucleic Acid Kit (Qiagen) according to the manufacturer's protocol. Briefly, serum samples were incubated with proteinase K and carrier RNA at 55° C. for 30 min in lysis buffer, and the circulating nucleic acids were bound to the silica membrane by applying vacuum pressure. After washing, the eluted samples were used for real-time RT-PCR. Primers of the mtDNA D-loop3 regions were used to quantify serum mtDNA. Anti-dsDNA antibodies were detected at 1:200 serum dilution using an ELISA kit (Alpha Diagnostic).

### Immune Complex Deposition in Kidney Glomeruli

Kidneys were harvested after perfusion with PBS from MRL/lpr mice. Frozen kidney sections were fixed in cold acetone for 20 min, washed, and blocked for 18 h at 4° C. with 4% BSA in PBS. To detect glomerular deposits, the sections were stained with FITC-conjugated anti-mouse C3 antibody (GC3-90E-Z, Immunology Consultants Laboratory) and Alexa Fluor 594-conjugated anti-Mouse IgG antibody (A-11020, Invitrogen) with Hoechst staining at 1:100 dilution (Life Technologies) for 1 h at room temperature. After washing with PBS, the tissues were mounted, and the slides were observed using a LSM880 laser confocal microscope. The fluorescence intensity score was determined after analyzing random images for each animal in a blinded manner.

### GEO Database Analysis

The microarray results of SLE (lupus) patient samples were obtained from Gene Expression Omnibus (GEO; National Center for Biotechnology Information, Bethesda, Me., USA; https://www.ncbi.nlm.nih.gov/geo/). Raw data were obtained from GEO accession no. GSE13887.

### Isolation of Normal-Density Granulocytes and Low-Density Granulocytes

Normal-density granulocytes (NDGs) were isolated from heparinized venous blood using a Ficoll-Paque gradient (GE Healthcare) with dextran (Sigma-Aldrich) sedimentation, followed by red blood cell lysis using hypotonic NaCl. From the PBMC layer, low-density granulocytes (LDGs) were isolated using a negative selection method. The cells were resuspended in RPMI 1640 medium for further characterization.

### Quantification and Visualization of NETs

NETs were induced in NDGs by incubating cells with calcium ionophore A23187 (25 μM) (Thermo Fisher) in RPMI 1640 medium for 2 h, and NETs were quantified using SYTOX fluorescent dye at 485/520 nm to quantify extracellular DNA. The fluorescence of PicoGreen (Life Technologies) at t=0 min was measured at 485/520 nm (emission/extinction) to quantify the total DNA. The fluorescence was quantified using a microplate reader (Synergy HTX; BIOTEK). NETs were also quantified by fluorescence microscopy. Briefly, the cells were attached to coverslip chambers, stimulated for 90 min at 37° C. with calcium ionophore, fixed with 4% paraformaldehyde overnight at 4° C., and permeabilized with 0.2% Triton X-100 for 10 min, followed by 0.5% gelatin for 20 min. The cells were stained with antibodies against human neutrophil elastase (ab21595, Abcam) for 2 h at room temperature, washed in PBS, and stained with Hoechst 33342 (Life Technologies) and Alexa Fluor 488 secondary antibody (A31570, Life Technologies) for 2 h at room temperature. After mounting, the cells were visualized with a LSM780 confocal microscope.

### Human Samples and Study Approval

Heparinized venous peripheral blood was obtained from SLE subjects or from healthy controls enrolled at the Clinical Center, National Institutes of Health. All individuals signed an informed consent form following IRB-approved protocols (NIH 94-AR-0066). SLE subjects fulfilled the revised American College of Rheumatology diagnostic criteria (Hochberg, 1997). Disease activity was determined using the SLEDAI-2K criteria (Hochberg (1997)). Individuals with recent or active infections were excluded.

### Statistical Analyses

Statistical comparisons between groups were performed using two-tailed unpaired Student's t-test and ANOVA with Tukey's post-hoc test for multiple comparisons using GraphPad Prism7 software (GraphPad). For the statistical analyses of human samples, the sample size was determined using similar patient numbers per experimental condition. The normality distribution of the sample sets was determined by d'Agostino and Pearson omnibus normality test. For sample sets with a Gaussian distribution, Student's two-tailed t-test, paired t-test, or Pearson's correlation coefficient analysis was performed. For the limited number of sample sets with a non-Gaussian distribution, Mann-Whitney U test was performed as applicable. Multiple comparisons with the same group in more than one analysis were adjusted using Bonferroni correction. All values are presented as the mean±SEM, and differences were considered statistically significant at p<0.05.

### Example 1

### Endonuclease G-Deficiency Increases Cytosolic mtDNA and Type I Interferon Signaling

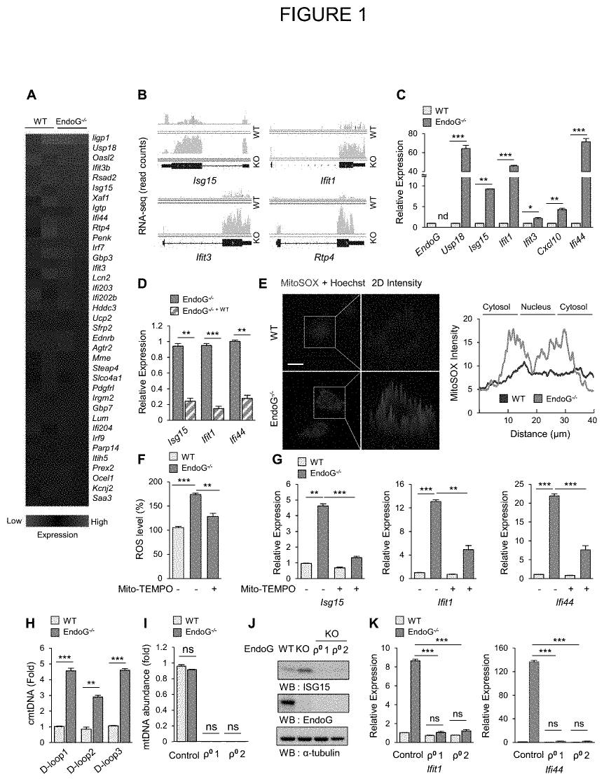

Modest mitochondrial stress caused by TFAM-deficiency in MEFs was shown to increase type I interferon signaling. In order to determine if this phenomenon is specific to TFAM-deficiency, type I interferon signaling was determined in Endonuclease G (EndoG)-deficient mouse embryo fibroblasts (MEFs). EndoG is a sugar-nonspecific nuclear-encoded mitochondrial endonuclease that is released from mitochondria during apoptosis and translocates to the nucleus to cleave chromosomal DNA. Its deficiency can lead to modest cardiac mitochondrial dysfunction and cardiac hypertrophy in older rodents and failure to degrade sperm or paternal mtDNA in invertebrates. To examine whether the modest mitochondrial stress in EndoG-deficient MEFs affects type I interferon response, RNAseq with wild-type (WT) and EndoG^{-/-} MEFs was performed. The results indicated that the mRNA levels of interferon-stimulated genes (ISGs), including Isg15, Ifit1 and Ifi44, were increased in EndoG^{-/-} MEFs (FIGS. 1A-1C). Restoring EndoG expression by reintroducing WT EndoG by stable transfection into EndoG^{-/-} MEFs (EndoG^{-/-+WT}) reduced ISG expression while knocking-down (KD) EndoG in WT MEFs elevated ISG expression, indicating that EndoG-deficiency, rather than other cellular differences between WT and EndoG^{-/-} MEFs, increased ISG expression (FIG. 1D). EndoG^{-/-} MEFs were also found to produce higher mROS level as shown by mitochondrial superoxide indicator mitoSOX (FIG. 1E). High mROS in EndoG^{-/-} MEFs was not due to reduction in antioxidant gene expression. To determine if the elevated mROS in EndoG^{-/-} MEFs was required for elevated ISG expression, MEFs were treated with mROS scavenger, mito-TEMPO for 3 days. As shown in FIGS. 1F-1G mito-TEMPO reduced ISG expression in EndoG^{-/-} MEFs accompanying mROS, indicating that mROS is required for upregulation of ISG expression in these cells. Cytosolic double-stranded DNA activates the cGAS-STING pathway to mediate ISG expression via the TBK1-IRF3 pathway. KD of EndoG in cGAS^{-/-} MEFs or IRF3/IRF7^{-/-} MEFs did not induce ISG expression, and KD of cGAS, STING, or TBK1 in EndoG^{-/-} MEFs decreased ISG expression in these cells. The cyclic GMP-AMP level (cGAMP) formed by cGAS, and the levels of IRF3 and phosphorylated STAT1 (in nucleus), were higher in EndoG^{-/-} MEFs. KD of another mitochondrial nuclease Exonuclease G (ExoG)²⁸ in EndoG^{-/-} MEFs further increased ISG expression. However, KD of ExoG in WT MEFs did not increase ISG expression. Taken together, these results suggested that the DNA sensing cGAS-STING pathway is involved in ISG expression in EndoG^{-/-} MEFs.

Since mtDNA is one of cGAS agonists, and EndoG^{-/-} MEFs have higher mROS than WT MEFs (FIG. 1F), it was postulated that mitochondria in EndoG^{-/-} MEFs may be more prone to release mtDNA. Indeed, as shown in FIG. 1H, cytosolic mtDNA (cmtDNA) was higher in EndoG MEFs even though the total mtDNA (FIG. 1I), mRNA encoded by the mitochondrial genes, as well as the expression levels of genes important for mitochondrial biogenesis and autophagy were similar between WT and EndoG^{-/-} MEFs. In order to demonstrate that elevated ISG expression was due to the activation of cGAS by elevated cmtDNA rather than by DNA from other sources (e.g. nucleus), mtDNA-depleted EndoG^{-/-} MEFs (ρ⁰ cells) were generated (FIG. 1I). In EndoG^{-/-} ρ⁰ MEFs, the ISG expression levels, as well as phosphorylation of TBK1 and IRF3, were significantly reduced compared to EndoG^{-/-} MEFs (FIGS. 1J-1K). If EndoG^{-/-} MEFs have higher type I interferon signaling, they should be more resistant to viral infection. To confirm this, WT and EndoG^{-/-} MEFs and EndoG^{-/-} MEFs which had EndoG expression restored by stable transfection (EndoG^{-/-+WT}) were infected with HSV-1 (Herpes Simplex Virus-1) expressing red fluorescent protein (RFP). The results indicated that the number of HSV-1 infected cells as measured by RFP expression and the number of particle forming units (PFU) produced by the infected cells were significantly lower in EndoG^{-/-} MEFs compared to the other two types of MEFs. Taken together, these findings indicated that EndoG-deficiency increases cmtDNA and the subsequent activation of the cGAS-STING pathway and type 1 IFN responses by increasing mitochondrial ROS.

### Example 2

### VDAC is Required for mtDNA Release

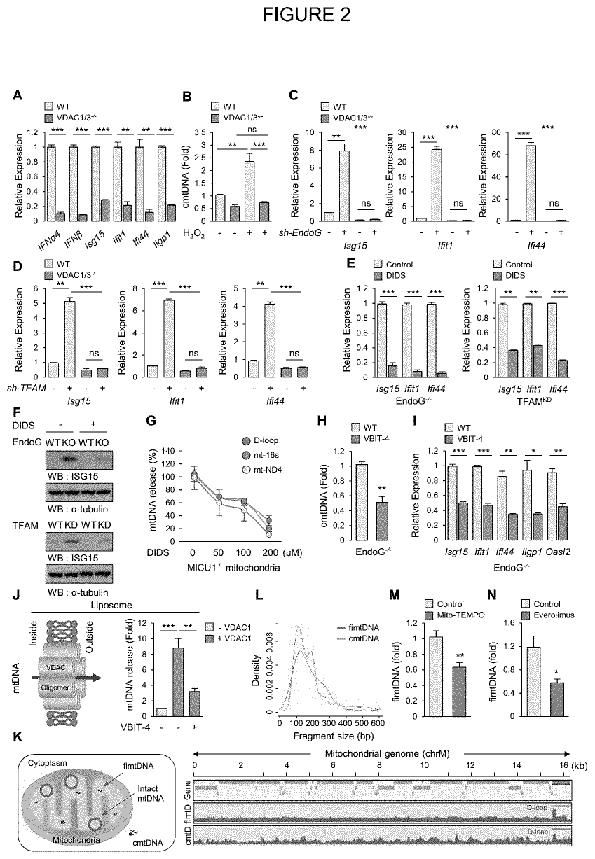

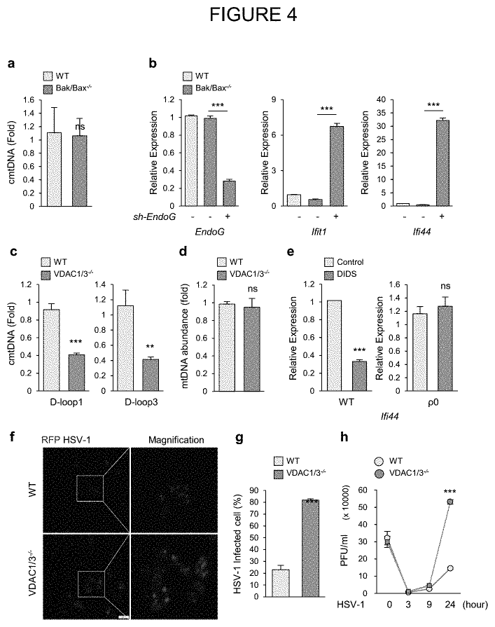

Bak and Bax are OMM proteins that can form macropores to facilitate mitochondrial herniation and mtDNA release during apoptosis is induced by Bak/Bax overexpression. However, it is not known whether these two proteins are required for mtDNA release in living cells, including those with modest mitochondrial stress. cmtDNA levels were measured in WT and Bak/Bax^{-/-} MEFs and the results indicated that cmtDNA levels were similar (FIG. 4A). In addition, ISG expressions were still induced when EndoG was knocked-down in Bak/Bax^{-/-} MEFs (FIG. 4B), suggesting that Bak/Bax may not be required for mtDNA release in living cells. The inventors then evaluated whether OMNI channel VDAC is required for mtDNA release. Unlike Bak/Bax^{-/-} MEFs, VDAC1/3^{-/-} MEFs had lower basal cmtDNA levels and ISG expressions compared to WT MEFs even though they had similar total mtDNA levels (FIG. 2A and FIGS. 4B-4C). Since ROS increases cmtDNA in EndoG^{-/-} MEFs (FIGS. 1F-1H), the inventors exogenously induced high ROS by treating WT and VDAC1/3^{-/-} MEFs with H₂O₂ and measured cmtDNA in these cells. As shown in FIG. 2B, H₂O₂ increased cmtDNA in WT MEFs but not in VDAC1/3^{-/-} MEFs. As is the case with EndoG deficiency, TFAM deficiency also increases cmtDNA and cGAS activation, leading to elevated ISG expression. The results indicated that KD of either EndoG or TFAM increased ISG expression in WT MEFs, but not in VDAC1/3^{-/-} MEFs FIGS. 2C-2D). To rule out that this difference was caused by differences between these cells which are unrelated to VDAC, EndoG^{-/-} and TFAM^{KD} MEFs were treated with the VDAC inhibitor DIDS (4,4′-Diisothiocyanatostilbene-2,2′-disulfonate) (Ben-Hail and Shoshan-Barmatz (2016)). As shown in FIGS. 2E-2F, DIDS inhibited ISG expression in both EndoG^{-/-} and TFAM^{KD} MEFs, thus confirming that VDAC is essential for mtDNA release and type I interferon signaling. Furthermore, the DIDS inhibited ISG expression in WT, but not in ρ⁰ cells indicating that mtDNA is required for VDAC function in ISG expression (FIG. 4E) rather than by DNA from other sources. The inventors than examined whether VDAC1/3-deficiency would reduce viral resistance, as VDAC was shown to be required for the type I interferon signaling. As expected, VDAC1/3^{-/-} MEFs were less resistant to HSV-1 infection (FIGS. 4F-4H). Therefore, VDAC, rather than Bak/Bax, appears to be the OMNI protein required for cmtDNA production in living cells.

### Example 3

### The N-Terminal of VDAC1 has a Role in mtDNA Release

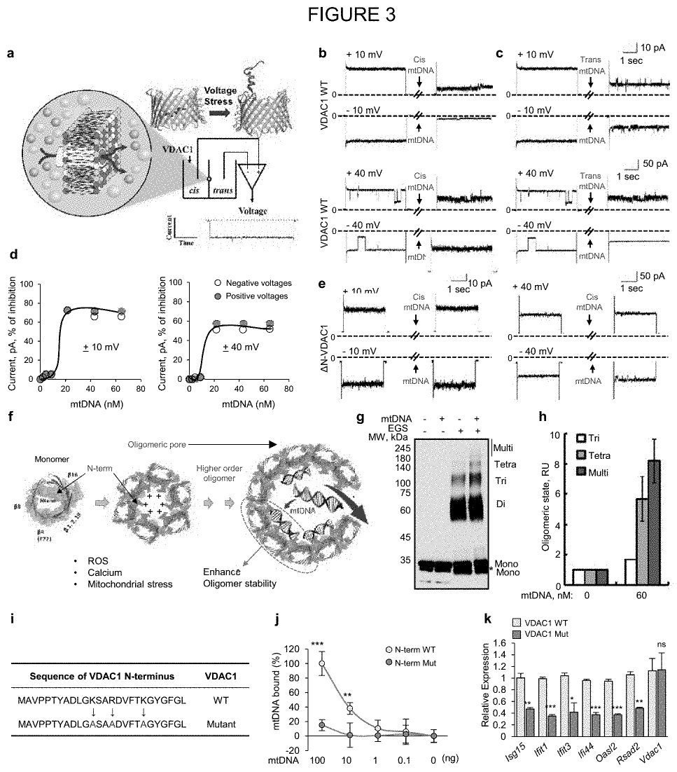

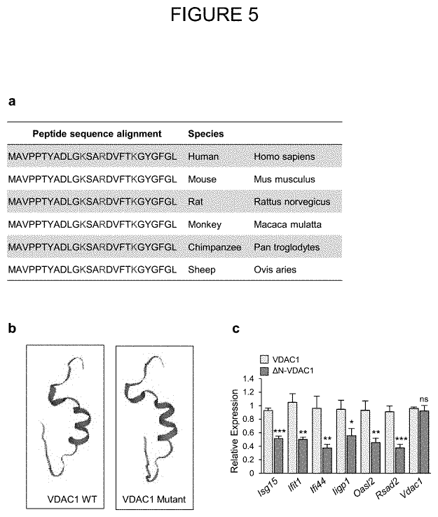

The N-terminus region is thought to translocate out of the VDAC1 pore when VDAC1 is in an oligomerized state, forming a pore significantly larger than that of a monomer (FIG. 3F). Unlike the β-barrel of VDAC, which is largely lipophilic, the N-terminus region, which is evolutionarily conserved is hydrophilic (FIG. 5A). Therefore, it is believed that the N-terminus region forms a hydrophilic ring around the oligomeric pore (FIG. 3F). It was speculated that the negatively charged backbone of mtDNA may interact with the hydrophilic residues in the N-terminus region of multiple VDAC1 molecules simultaneously and act as a scaffold to stabilize oligomers (FIG. 3F). To evaluate this possibility, VDAC1 was incubated with protein-protein cross-linking agent ethylene glycol bis(succinimidylsuccinate) (EGS) either in the presence or absence of mtDNA. As shown in FIGS. 3G-3H, mtDNA did not increase the formation of VDAC1 dimers but significantly increased the formation of trimers and higher order oligomers. The N-terminus region contains three positively-charged amino acid residues (K12, R15, K20) that could interact with the negatively-charged backbone of mtDNA (FIG. 3I). Indeed, 26 amino acid VDAC1 N-terminal peptide pulled-down the mtDNA fragment in dose-dependent manner but not VDAC1 N-terminal mutant peptide in which K12, R15, K20 were changed to Ala (A) (FIG. 3J). Structural prediction analysis indicated that these mutations did not significantly change the overall structure of the N-terminal peptide (FIG. 5B). The ISG expression was then determined in VDAC1/3^{-/-} MEFs with restored expression of either WT VDAC1, the mutant VDAC1 (FIG. 3I) or ΔN-terminus VDAC1, after treatment with H₂O₂. The results indicated that MEFs expressing either the mutant VDAC1 (FIG. 3K) or ΔN-terminus VDAC1 (FIG. 5C) had significantly reduced ISG expression compared to those expressing WT VDAC1, suggesting that the N-terminal region is important for both interacting with mtDNA and activating the cGAS pathway.

### Example 4

### VDAC has a Ca²⁺ Flux-Independent Role in mtDNA Release

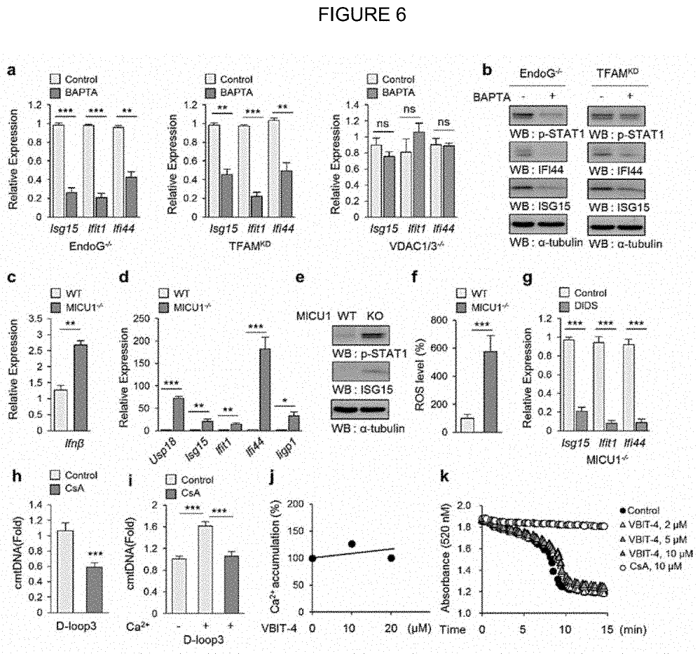

VDAC functions are interlinked with mitochondrial Ca²⁺ and ROS: VDAC can control their flux across the OMNI and they in turn can increase VDAC expression and oligomerization. Therefore, it was hypothesized that Ca²⁺ and ROS may regulate mtDNA release in living cells. Treatment with Ca²⁺ chelator BAPTA decreased ISG expression in EndoG^{-/-} MEFs or TFAM^{KD} MEFs, but not in VDAC1/3^{-/-} MEFs (FIGS. 6A-6B). To further investigate the role of Ca²⁺ in interferon-signaling in living cells, MEFs deficient in MICU1, a Ca²⁺-gatekeeper that prevents Ca²⁺ overload in the mitochondrial matrix, were used. Both interferon-signaling and ROS level were increased in MICU1^{-/-} MEFs (FIGS. 6C-6F), but treatment with DIDS abrogated ISG induction in these cells, suggesting that VDAC is important for Ca²⁺-induced type 1 IFN responses in living cells (FIG. 6G).

In order to determine if PTP opening is important for basal mtDNA release, both WT MEFs and mitoplasts were treated with CysA and the results indicated that CysA decreased mtDNA release (FIGS. 6H-6I). Therefore, it was postulated that VDAC may be increasing mtDNA release in living cells merely by increasing mitochondrial Ca²⁺ and promoting PTP formation. To examine this possibility, mitochondria from MICU1^{-/-} MEFs, which already have elevated Ca²⁺ and ROS levels (FIG. 6F), were isolated and incubated in a buffer lacking Ca²⁺ either with or without DIDS. As shown in FIG. 2G, DIDS decreased in a concentration-dependent manner mtDNA released during incubation, suggesting that VDAC also has a Ca²⁺ flux-independent function in mtDNA release.

To further confirm this finding, a highly potent VDAC oligomerization inhibitor VBIT-4 which interacts directly with VDAC and specifically inhibits VDAC oligomerization capacity was used. First, the effect of VBIT-4 on Ca²⁺ uptake and PTP opening in purified mitochondria was evaluated and it was found that VBIT-4 did not prevent either Ca²⁺ uptake or PTP opening (FIGS. 6J-6K). However, treatment of EndoG^{-/-} MEFs with VBIT-4 decreased cmtDNA and ISG expression (FIGS. 2H-2I) indicating that VBIT-4 can decrease cmtDNA release without directly inhibiting either Ca²⁺ uptake or PTP opening.

In order determine if VDAC is sufficient to allow the passage of mtDNA through OMM, mtDNA fragments were loaded into liposomes either with or without VDAC1. After incubation, mtDNA release from liposomes was then measured by real-time RT-PCR. As shown in FIG. 2J, the presence of VDAC1 in liposomes significantly increased mtDNA release, but VBIT-4 decreased the release. Therefore, even though VDAC can control PTP opening by serving as the major channel for Ca²⁺ uptake, these findings revealed that VDAC oligomerization can also promote mtDNA release independent of VDAC function in Ca²⁺ flux and PTP opening.

As the intact mtDNA is tethered to the inner mitochondrial membrane (IMM) in nucleoid complexes and is generally not diffusible, it was suspected that free intra-mtDNA fragments (fimtDNA) pre-exist under normal conditions prior to passing through the membranes. Conditions that have high mtDNA release such as cells with mitochondrial dysfunction (e.g., EndoG^{-/-} and TFAM^{KD}) or apoptosis are easier for studying mtDNA release. However, results from these cells are also difficult to interpret because mitochondrial dysfunction can also alter cellular division rates, which can then alter mitochondrial division rates, and high ROS levels and activation of cytosolic nucleases, which can accompany apoptosis, may further damage mtDNA. Also, activation of PTP itself may lead to mtDNA damage because activation of PTP can increase ROS, which has been reported to break mtDNA in certain cells. To minimize the confounding variables, fimtDNA was evaluated in WT MEFs under normal growing conditions. To do this, mitochondria purified from WT MEFs were treated with cytoskeleton (CSK) buffer, which permeabilizes mitochondrial membranes but leaves the mitochondrial nucleoids intact. After permeabilization, the mtDNA released into the supernatant (fimtDNA) was isolated and sequenced without first cleaving it. The results indicated that the sequences corresponding to a region within the D-loop in mitochondrial genome was significantly more abundant than those corresponding to the other regions (FIG. 2K). A similar analysis of cmtDNA from WT MEFs showed that although the sequences corresponding to the same D-loop region was slightly more abundant than those from the other regions, the difference was not as pronounced as those in fimtDNA. The size distribution analysis of fimtDNA and cmtDNA, which excluded the sequences that had 100% homology to both mitochondrial and nuclear genomes, indicated that the peak size was relatively short for both (˜110 bp) (FIG. 2L). It was hypothesized that fimtDNA may be most abundant in the subpopulation of oxidatively damaged mitochondria that have not been eliminated by autophagy. To test this, fimtDNA in mitochondria isolated from cells treated with mitochondrial antioxidant mito-TEMPO (FIG. 2M) or mTORC1 inhibitor and a rapalog everolimus, which increases autophagy was measured (FIG. 2N). In agreement with this hypothesis, both treatments decreased fimtDNA.

### Example 5

### Mitochondrial DNA Interacts with VDAC

The next aim was to determine whether VDAC may also have a role in mtDNA release via direct interaction. One method for detecting direct interaction was by reconstituting purified mitochondrial VDAC1 into a planar lipid bilayer (PLB) and measuring the effect of DNA on channel conductance under voltage clamp conditions (FIG. 3A). For that aim, a fragment that was derived from the D-loop region of mtDNA was utilized. Mitochondrial DNA inhibited VDAC1 conductance after exposure to high voltage (60 mV) but not to low voltage (10 mV). This voltage-dependence is known to be important because evidence suggested that high voltage exposes the N-terminus region by inducing its translocation out of the VDAC pore, potentially allowing its interaction with mtDNA. Indeed, mtDNA-VDAC1 interaction measured by micro-scale thermophoresis (MST), which is performed without prior exposure to high voltage, showed no interaction, whereas VBIT-4 did show interaction. VDAC1 conductance at both ±10 mV and ±40 mV was inhibited by mtDNA after a prior exposure to 60 mV irrespective of the side of the PLB to which the mtDNA was added, Cis (cytoplasm side, FIG. 3B) or Trans (intermembrane space side, FIG. 3C) with the IC₅₀ of 10-13 nM (FIG. 3D). However, mtDNA did not inhibit the channel conductance of ΔN-terminus VDAC1 (N-terminal truncation mutant) (FIG. 3E), indicating that mtDNA can interact with the N-terminus region of VDAC1.

### Example 6

### VBIT-4 Ameliorates Lupus-Like Disease

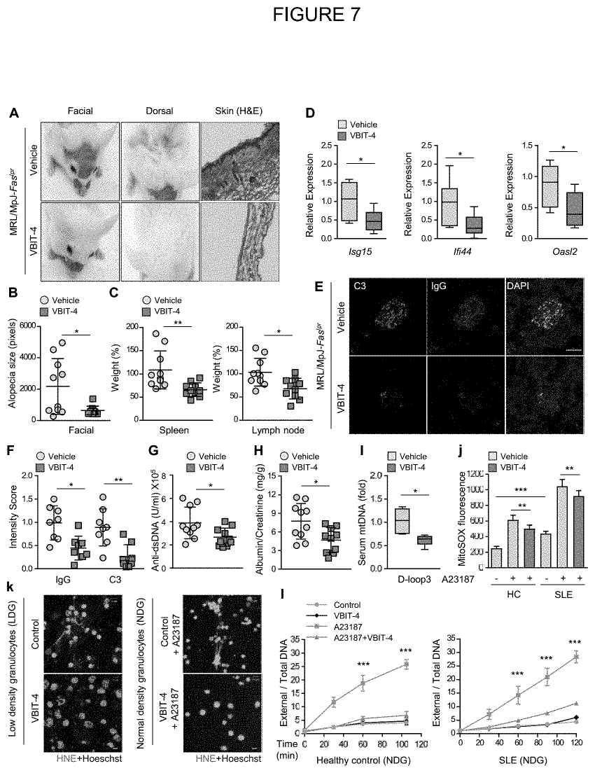

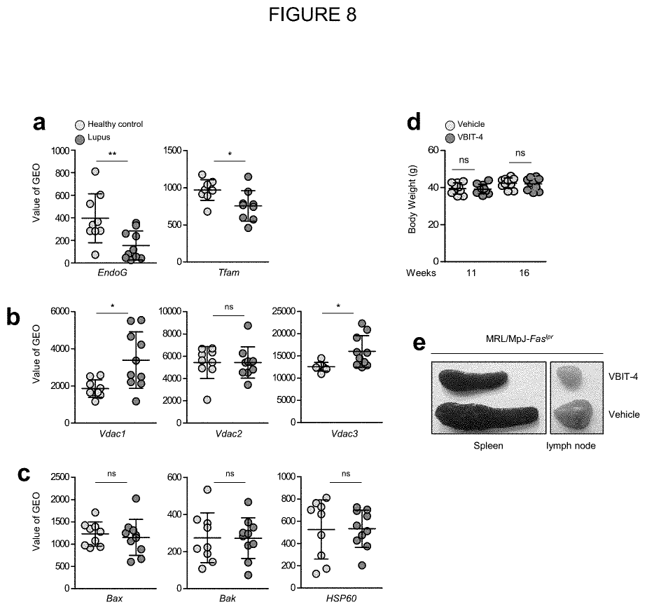

Using Gene Expression Omnibus (GEO) analysis, the inventors found that mRNA expression of VDAC1/3 was elevated in lupus patients but mRNA expression of EndoG and TFAM was reduced. Expression levels of VDAC2, HSP60, Bak and Bax were not changed in lupus patients. These findings combined with the observation that VDAC promoted type 1 IFN response in EndoG and TFAM deficiency (FIGS. 2C-2D), suggested that inhibiting VDAC oligomerization with VBIT-4 may affect the clinical course of lupus. In order to test the effect of VBIT-4 in lupus, MRL/lpr lupus-prone mice were treated with VBIT-4 for 5 weeks. VBIT-4 treatment did not cause any mortality or change in body weight (FIG. 8D). VBIT-4 treatment blocked the development of skin lesions and the thickening of the epidermis that accompanies leukocyte infiltration, and suppressed alopecia of face and dorsal (FIGS. 7A-7B). VBIT-4 treatment also reduced the weight of spleen and lymph node (FIGS. 7C and 8E) and significantly diminished ISG induction, renal immune complex deposition, serum anti-dsDNA, proteinuria and cell-free mtDNA compared with vehicle-treated MRL/lpr mice (FIGS. 7D-71).

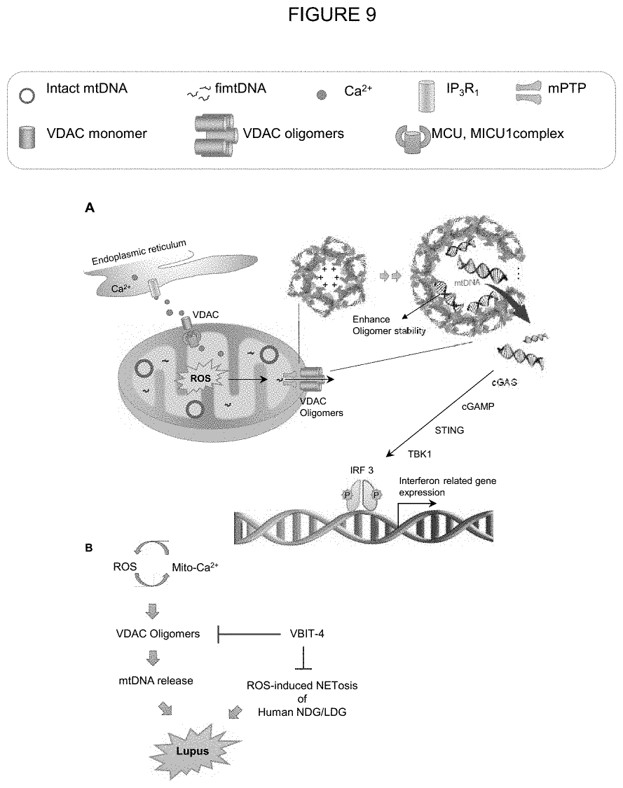

One potential source of cell-free mtDNA in MRL/lpr mice is neutrophil extracellular traps (NETs), which are networks of processed chromatin structures, including genomic and mitochondrial DNA. They are released from neutrophils in a cell-death process called NETosis to entangle and kill microbes but are also implicated in autoimmunity such as lupus. Since mitochondrial ROS increases during NETosis induced by certain triggers such as A23187 Ca²⁺ ionophore, the effect of VBIT-4 on mitochondrial ROS during NETosis was evaluated. Indeed, as shown in FIG. 7J, VBIT-4 decreased A23187-induced mitochondrial ROS in neutrophils from healthy controls as well as those from lupus patients. NETosis was then induced in low-density granulocytes (LDG), a distinct class of pro-inflammatory and NETosis-prone neutrophils in SLE patients, and normal-density granulocytes (NDG) isolated from lupus patients and healthy controls, in the presence of VBIT-4. As shown in FIG. 7K, VBIT-4 strongly inhibited NETosis in both LDG and NDG from lupus patients. Similarly, VBIT-4 strongly inhibited NETosis in NDG from healthy controls and lupus patients (FIG. 7L). VBIT-4, which did not cause any mortality or changes in the body weight (FIG. 8D), blocked the development of skin lesions and the thickening of the epidermis that accompanies leukocyte infiltration and suppressed facial and dorsal alopecia (FIGS. 7A-7B). VBIT-4 also decreased the weight of the spleen and lymph nodes (FIGS. 7C and 8E). Taken together, these findings indicate that VDAC oligomerization promotes NETosis, an important trigger of autoimmunity, and lupus-like disease in MRL/lpr mice, and that VBIT-4 inhibited this process. The role of VDAC oligomerization in interferon gene expression and the inhibitory effect of VBIT-4 on VDAC oligomerization and NETosis are presented in FIGS. 9A-9B, respectively. The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

While certain features of the invention have been described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. A method for slowing the progression of or treating an autoimmune disease comprising administering to a subject in need of such treatment a pharmaceutical composition comprising a therapeutically effective amount of a VDAC inhibiting compound.

2. The method of claim 1, wherein said VADC inhibiting compound is of the general Formula (I): 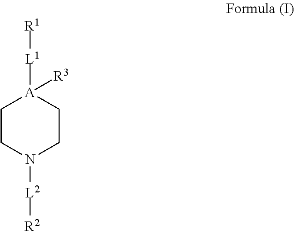
wherein:
A is carbon (C) or nitrogen (N);
R³ is absent, a hydrogen, an unsubstituted or substituted amide, or a heteroalkyl comprising 3-12 atoms apart from hydrogen atoms, wherein at least one of said 3-12 atoms is nitrogen, sulfur or oxygen, wherein when A is nitrogen (N), R³ is absent;
L¹ is absent or is an amino linking group —NR⁴—, wherein R⁴ is hydrogen, a C₁₋₅-alkyl, a C₁₋₅-alkylene or a substituted alkyl —CH₂R, wherein R is a functional group selected from the group consisting of hydrogen, halo, haloalkyl, cyano, nitro, hydroxyl, alkyl, alkenyl, aryl, alkoxyl, aryloxyl, aralkoxyl, alkylcarbamido, arylcarbamido, amino, alkylamino, arylamino, dialkylamino, diarylamino, arylalkylamino, aminocarbonyl, alkylaminocarbonyl, arylaminocarbonyl, alkylcarbonyloxy, arylcarbonyloxy, carboxyl, alkoxycarbonyl, aryloxycarbonyl, sulfo, alkylsulfonylamido, alkyl sulfonyl, arylsulfonyl, alkyl sulfinyl, arylsulfinyl and heteroaryl;
R¹ is an aromatic moiety, which is optionally substituted with one or more of Z;
Z is independently at each occurrence a functional group selected from the group consisting of, hydrogen, halo, haloalkyl, haloalkoxy, perhaloalkoxy or C₁₋₂-perfluoroalkoxy, cyano, nitro, hydroxyl, alkyl, alkenyl, aryl, alkoxyl, aryloxyl, aralkoxyl, alkylcarbamido, arylcarbamido, amino, alkylamino, arylamino, dialkylamino, diarylamino, arylalkylamino, aminocarbonyl, alkylaminocarbonyl, arylaminocarbonyl, alkylcarbonyloxy, arylcarbonyloxy, carboxyl, alkoxycarbonyl, aryloxycarbonyl, sulfo, alkylsulfonylamido, alkyl sulfonyl, aryl sulfonyl, alkylsulfinyl, arylsulfinyl and heteroaryl;
L² is a linking group, such that when A is nitrogen (N), L² is a group consisting of 4-10 atoms, apart from hydrogen atoms, optionally forming a ring, whereof at least one of the atoms is nitrogen, said nitrogen forming part of an amide group; and when A is carbon (C), then L² is selected from C₁₋₄ alkylene or a group consisting of 4-10 atoms, apart from hydrogen atoms, optionally forming a ring, whereof at least one of the atoms is nitrogen, said nitrogen forming part of an amide group;
R² is a phenyl or a naphthyl, optionally substituted with a halogen;
or an enantiomer, diastereomer, mixture or salt thereof.

3. The method of claim 2, wherein A is nitrogen (N), and said linking group L² is selected from the group consisting of a C₄₋₆-alkylamidylene and a pyrrolidinylene, said linking group optionally substituted with a substituent comprising an alkyl, a hydroxy, an oxo or a thioxo group, optionally wherein L² is selected from the group consisting of butanamidylene, N-methylbutanamidylene, N,N-dimethylbutanamidylene, 4-hydroxybutanamidylene, 4-oxobutanamidylene, 4-hydroxy-N-methylbutanamidylene, 4-oxo-N-methylbutanamidylene, 2-pyrrolidonyl, pyrrolidine-2,5-dionylene, 5-thioxo-2-pyrrolidinonylene and 5-methoxy-2-pyrrolidinonylene, and optionally wherein when L² is butanamidylene, N-methylbutanamidylene, N,N-dimethylbutanamidylene, 4-hydroxybutanamidylene, 4-oxobutanamidylene, 4-hydroxy-N-methylbutanamidylene or 4-oxo-N-methylbutanamidylene, the carbon (C) in third position of the butanamide moiety is bonded and the nitrogen (N) of the butanamide moiety is bonded to R²; or wherein when L² is 2-pyrrolidone, pyrrolidine-2,5-dione, 5-thioxo-2-pyrrolidone or 5-methoxy-2-pyrrolidone, a carbon (C) of the pyrrolidine moiety is bonded to the nitrogen (N) of the piperazine ring and the nitrogen (N) of the pyrrolidine moiety is bonded to R².

4. (canceled)

5. (canceled)

6. The method of claim 2, wherein A is carbon (C), R³ is a substituted amide group, and L² is methylene.

7. The method of claim 2, wherein the compound is of general Formula (Ia): 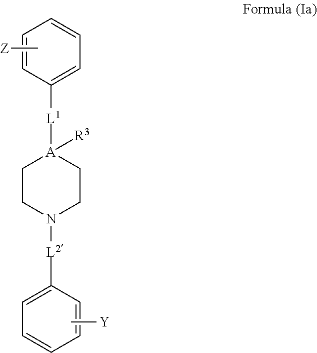
wherein:
A, R³, Z and L¹ as defined in claim 2,
L^{2′} is a linking group selected from the group consisting of an C₄-alkylamidylene, an C₅-alkylamidylene and an C₆-alkylamidylene, optionally substituted with one or two of alkyl, hydroxy, oxo or thioxo group; and
Y is a halogen;
or an enantiomer, diastereomer, mixture or salt thereof, optionally wherein carbon (C) at position 3 of alkylamidylene L^{2′} is bonded to the nitrogen (N) of the piperazine ring or of the piperidine ring, and the nitrogen (N) of said alkylamidylene L^{2′} is bonded to the phenyl group.

8. (canceled)

9. The method of claim 2, wherein the compound is of the general Formula (Ib): 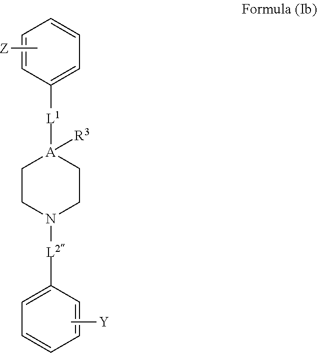
wherein:
A, R³, and Z are as defined in claim 2,
L¹ is absent;
L^{2″} is a pyrrolidinylene linking group, optionally substituted with one or two of alkyl, hydroxy, oxo or thioxo group;
Y is halogen;
or an enantiomer, diastereomer, mixture or salt thereof.

10. The method of claim 9, wherein L^{2″} is selected from 2-pyrrolidonylene, pyrrolidine-2,5-dionylene, 5-thioxo-2-pyrrolidinonylene and 5-methoxy-2-pyrrolidinonylene, and optionally wherein a carbon (C) atom of the pyrrolidinyl moiety L2″ is bonded to the nitrogen (N) of the piperazine ring or the piperidine ring and the nitrogen (N) of the pyrrolidinyl moiety is bonded to the phenyl group.

11. (canceled)

12. The method of claim 2, wherein the compound is of the general Formula (Ic): 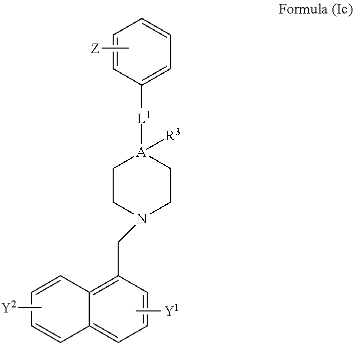
wherein:
A, R³, and Z are as defined in claim 2,
L¹ is —NH—;
Y¹ and Y² are each independently absent or a halogen;
or an enantiomer, diastereomer, mixture or salt thereof.

13. The method of claim 12, wherein R³ is —C(O)NHCH₂C(O)OH group.

14. The method of claim 12, wherein Z is C₁₋₂-alkoxy or C₁₋₂-perfluoroalkoxy.

15. The method of claim 2, wherein the compound is of the general Formula (Id): 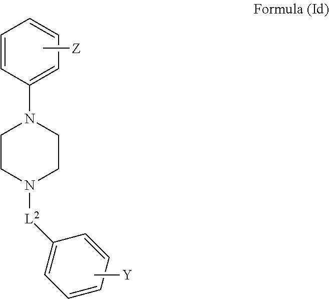
wherein
Z is C₁₋₂-perfluoroalkoxy, and Y is halogen.

16. The method of claim 15, wherein the compound having the Formula 1: 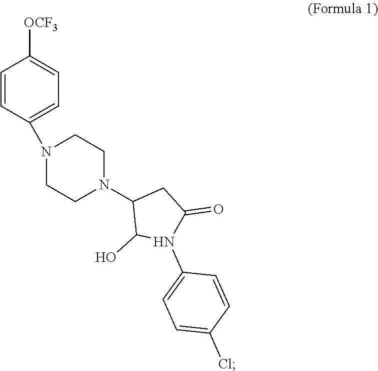 [SMILES: O=C1CC(N2CCN(c3ccc(OC(F)(F)F)cc3)CC2)C(O)N1c1ccc(Cl)cc1]
or an enantiomer, diastereomer, mixture or salt thereof.

17. The method of claim 13, wherein the compound having the Formula 3: 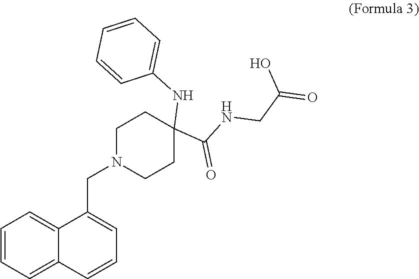 [SMILES: O=C(O)CNC(=O)C1(Nc2ccccc2)CCN(Cc2cccc3ccccc23)CC1]
or an enantiomer, diastereomer, mixture or salt thereof.

18. The method of claim 2, wherein the compound is of Formula (IIa): 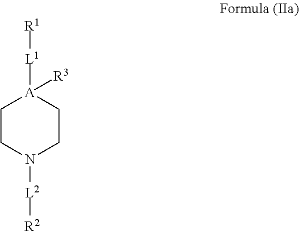
wherein:
A is carbon (C);
R³ is hydrogen or heteroalkyl chain comprising 3-12 atoms, apart from hydrogen atoms, wherein at least one is a heteroatom, selected from nitrogen, sulfur and oxygen;
L¹ is an amino linking group —NR⁴—, wherein R⁴ is hydrogen, a C₁₋₅-alkyl, a C₁₋₅-alkylene or a substituted alkyl —CH₂R, wherein R is a functional group selected from hydrogen, halo, haloalkyl, cyano, nitro, hydroxyl, alkyl, alkenyl, aryl, alkoxyl, aryloxyl, aralkoxyl, alkylcarbamido, arylcarbamido, amino, alkylamino, arylamino, dialkylamino, diarylamino, arylalkylamino, aminocarbonyl, alkylaminocarbonyl, arylaminocarbonyl, alkylcarbonyloxy, arylcarbonyloxy, carboxyl, alkoxycarbonyl, aryloxycarbonyl, sulfo, alkylsulfonylamido, alkyl sulfonyl, aryl sulfonyl, alkylsulfinyl, arylsulfinyl or heteroaryl;
when R³ is heteroalkyl group comprising 3-12 atoms, apart from hydrogen atoms, then L¹ forms a ring with R³;
R¹ is an aromatic moiety, which is optionally substituted with one or more of C₁₋₂-alkoxy, and/or C₁₋₂-perfluoroalkoxy;
L² is a linking group consisting of 4-10 atoms, apart from hydrogen atoms, optionally forming a ring, whereof at least one of the atoms is nitrogen, said nitrogen forming part of an amide group or L² is C₁₋₅-alkyl or C₁₋₅ alkylene; said linking group L² bonds piperidine or piperazine moiety at nitrogen (N) atom; and
R² is an aryl, optionally substituted with halogen, optionally when R² is a phenyl it is substituted with halogen, further optionally when R² is naphthyl, L² is an alkylenyl group;
or an enantiomer, diastereomer, mixture or salt thereof.

19. The method of claim 18, wherein the compound of Formula (IIa) has the Formula 10: 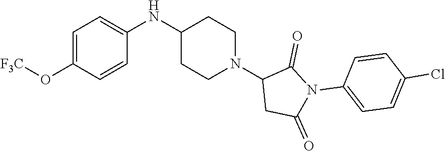 [SMILES: O=C1CC(N2CCC(Nc3ccc(OC(F)(F)F)cc3)CC2)C(=O)N1c1ccc(Cl)cc1]
or has the Formula 11:

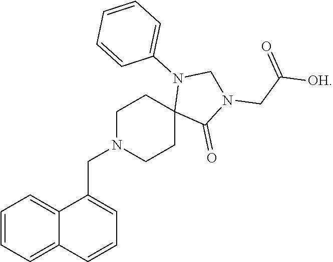 [SMILES: O=C(O)CN1CN(c2ccccc2)C2(CCN(Cc3cccc4ccccc34)CC2)C1=O]

20. (canceled)

21. The method of claim 1, wherein said VDAC inhibiting compound is a peptide derived from or corresponding to amino acids residues 1-26 of human VDAC1 N-terminal domain (SEQ ID NO:1), comprising: (i) one or more mutations compared to the SEQ ID NO:1, (ii) a truncation of one or more amino acids compared to said SEQ ID NO:1, or a combination thereof, optionally wherein said peptide is a peptide of 1-25 amino acids comprising a contiguous sequence derived from amino acids residues 1-26 of human VDAC1 N-terminal domain comprising the amino acid sequence: MAVPPTYADLGKSARDVFTKXYXFX (SEQ ID NO:2), wherein X is any amino acid other than glycine, and optionally wherein said peptide comprises an amino acid sequence selected from the group consisting of: SEQ ID Nos.:4-13.

22. (canceled)

23. (canceled)

24. The method of claim 1, wherein said VDAC1 inhibiting compound is a VDAC silencing oligonucleotide molecule or a construct comprising a VDAC silencing oligonucleotide molecule, optionally wherein said VDAC1 silencing oligonucleotide molecule comprises a nucleic acid sequence comprising at least 15 contiguous nucleotides identical to SEQ ID NO:17, an mRNA molecule encoded by SEQ ID NO:17, or a sequence complementary thereto, and optionally wherein the VDAC1 silencing oligonucleotide molecule is selected from the group consisting of SEQ ID NO: 18-25.

25. (canceled)

26. (canceled)

27. The method of claim 1, wherein said autoimmune disease is selected from the group consisting of: an autoimmune disease involving a systemic autoimmune disorder, and autoimmune disease involving a single cell-type autoimmune disorder, and optionally wherein said autoimmune disease involving a systemic autoimmune disorder is selected from the group consisting of: systemic lupus erythematosis (SLE), rheumatoid arthritis (RA), Sjogren's syndrome, systemic sclerosis, multiple sclerosis (MS), and bullous pemphigoid, and optionally wherein said autoimmune disease involving a single cell-type autoimmune disorder is selected from the group consisting of: Hashimoto's thyroiditis, autoimmune hemolytic anemia, autoimmune atrophic gastritis, autoimmune encephalomyelitis, autoimmune orchitis, Goodpasture's disease, autoimmune thrombocytopenia, myasthenia gravis (MG), Graves' disease, primary biliary cirrhosis, and membranous glomerulopathy.

28. (canceled)

29. The method of claim 27, wherein: said autoimmune disease involving a systemic autoimmune disorder is SLE; or said autoimmune disease involving a systemic autoimmune disorder is MS, and the subject has no depression or any other mood disorder.

30. (canceled)

31. (canceled)

32. The method of claim 1, wherein said pharmaceutical composition further comprises a pharmaceutically acceptable excipient, and optionally wherein said pharmaceutical composition is administered orally or parenterally.

33. (canceled)
